(19) United States
(12) Patent Application Publication
(10) Pub. No.: US 2025/0367384 A1
(43) Pub. Date: Dec. 4, 2025

(54) MEDICAL INJECTIONS AND RELATED DEVICES AND METHODS

(71) Applicant: Gilead Sciences, Inc., Foster City, CA (US)

(72) Inventors: Stephan Edward Augustyn, Cambridge (GB); Walter Goodwin, San Carlos, CA (US); William James Kirby, Cambridge (GB); Finlay Knops-McKim, Cambridge (GB); Daniel Milton Meruz, Cambridge (GB); James Paul Oberhauser, Saratoga, CA (US); Adam Toner, Harpenden (GB); Alasdair Young, Foster City, CA (US)

(21) Appl. No.: 19/206,223

(22) Filed: May 13, 2025

Related U.S. Application Data
(60) Provisional application No. 63/647,307, filed on May 14, 2024.

(51) Int. Cl. A61M 5/315; A61K 9/00; A61K 31/4439; A61K 47/10; A61M 5/20

(52) U.S. Cl. CPC A61M 5/31541; A61K 9/0019; A61K 31/4439; A61K 47/10; A61M 5/2046; A61M 5/31511; A61M 2205/3368; A61M 2205/3576; A61M 2205/581

(57) ABSTRACT

Systems, devices, and methods include an autoinjector including a housing; a needle arranged at a distal end of the housing; a container disposed within the housing and configured to contain medicament; and a plunger slidably disposed within the container. A plunger rod is configured to push the plunger through the container to dispense the medicament through the needle when the container contains medicament. The plunger rod includes one or more optical transmission regions. A mechanism is configured so that a dispensing movement of the plunger rod causes the mechanism to generate a first optical signal and transmit the first optical signal through at least one of the one or more optical transmission regions; and an optical sensor is configured to detect the first optical signal generated by the mechanism.

### CROSS REFERENCE TO RELATED APPLICATIONS

This application claims priority to U.S. Provisional Application No. 63/647,307, filed May 14, 2024, the entire content of which application is hereby incorporated by reference in its entirety.

### TECHNICAL FIELD

This disclosure relates to medical injections and related devices and methods.

### BACKGROUND

An injection typically refers to the act of administering a liquid (e.g., a drug) into a patient's body tissue. Injecting medicament into a patient can allow the medicament to be absorbed relatively rapidly.

### SUMMARY

The present systems, devices, and methods can sense a dispensing movement of a plunger rod within an autoinjector. For example, one or more optical transmission regions on the plunger rod of the autoinjector can enable optical signals to be transmitted and received by a sensing assembly to track the dispensing movement of the plunger rod. The signals can include voltage or other electrical measurements that indicate that an optical transmission region has reached a certain location within the autoinjector or that a certain number of optical transmission regions has progressed through the autoinjector. The devices can include a sensing assembly that can determine that the plunger rod has completed the dispensing movement after receiving a predetermined number of signals. The sensing assembly can also track the dispensing movement of the plunger rod. Tracking the dispensing movement of the plunger rod can be advantageous because the dispensing movement of the plunger rod corresponds to the amount of medicament delivered to the subject (e.g., a patient). If the plunger rod does not complete the dispensing movement, then the full dose of medicament is not injected into the patient. The sensing assembly can be used to determine that the full dose is injected into the patient by tracking the dispensing movement. Tracking movement of the plunger rod throughout the dispensing movement can be advantageous for determining how much of a medicament has been delivered and whether the amount of medicament injected exceeds a minimum dose volume threshold for medicament efficacy.

The present systems, devices, and methods can also sense whether a needle of the autoinjector is inserted into a patient to a sufficient depth and/or whether the insertion depth is maintained during delivery of the medicament. For example, an electrical contact between a needle guard and a particular location in the autoinjector can determine whether the needle has been inserted to a sufficient depth. Determining that the needle is sufficiently inserted into the patient can be advantageous because if the needle is not sufficiently inserted into the patient, the patient may not receive the medicament properly.

The present disclosure relates to a composition comprising lenacapavir or a pharmaceutically accepted salt thereof for use in the prevention or treatment of HIV, where the lenacapavir is administered by an autoinjector according to this disclosure. The present disclosure also relates to a use of lenacapavir or a pharmaceutically accepted salt thereof for the manufacture of a medicament for the prevention or treatment of HIV, where the lenacapavir is administered by an autoinjector according to the disclosure. In a first aspect of the invention, the present disclosure encompasses an autoinjector including a housing; a needle arranged at a distal end of the housing; a container disposed within the housing and configured to contain medicament; a plunger slidably disposed within the container; a plunger rod configured to push the plunger through the container to dispense the medicament through the needle when the container contains medicament, where the plunger rod includes one or more optical transmission regions; an optical emitter configured to emit a first optical signal through at least one of the one or more optical transmission regions of the plunger rod; and an optical receiver configured to receive the first optical signal, where a dispensing movement of the plunger rod enables transmission of the first optical signal from the optical emitter through the at least one of the one or more optical transmission regions and to the optical receiver.

According to the first aspect of the invention, the first optical signal may be indicative of dose progression.

According to the first aspect of the invention, the container may contain the medicament. Additionally or alternatively, the medicament may comprise lenacapavir or a pharmaceutically accepted salt thereof.

According to the first aspect of the invention, the autoinjector may further comprise a gas canister assembly configured to release pressurized gas which, when released, provides a force acting on the plunger rod to push the plunger through the container.

According to the first aspect of the invention, the at least one of the one or more optical transmission regions may include an aperture disposed within the plunger rod. Alternatively, the at least one of the one or more optical transmission regions may include an optically transparent region disposed within the plunger rod. The plunger rod may be configured to transmit a plurality of optical signals during the dispensing movement of the plunger rod. The plunger rod may include a plurality of optical transmission regions. Additionally or alternatively, the first optical signal may be configured to indicate completion of the dispensing movement of the plunger rod. A first optical transmission region of the plurality of optical transmission regions may be configured to indicate dose start of the medicament, and/or a last optical transmission region of the plurality of optical transmission regions may be configured to indicate dose end of the medicament. Additionally or alternatively, each optical transmission region of the plurality of optical transmission regions may be configured to transmit a corresponding optical signal during the dispensing movement of the plunger rod.

The autoinjector according to the first aspect of the invention may, additionally or alternatively, include an audible clicker configured to produce one or more audible clicks during the dispensing movement of the plunger rod. The audible clicker may include a ring surrounding the plunger rod, and the ring includes a deflectable protrusion configured to contact the plunger rod and produce the one or more audible clicks. Optionally, the plunger rod may include a ridged surface configured to contact the deflectable protrusion of the audible clicker. Further optionally, each ridge of the ridged surface may be configured to cause the deflectable protrusion to deflect during the dispensing movement of the plunger rod.

According to the first aspect of the invention, each of the optical emitter and the optical receiver may be, independently, disposed between a proximal end and a distal end of the housing. The optical emitter may include an infrared emitter, and/or the optical receiver may include an infrared receiver. Some embodiments according to the first aspect of the invention include an optical reflector configured to provide an optical path between the optical emitter and the optical receiver. Optionally, the optical reflector is disposed on a first surface within the housing, and the optical emitter and the optical receiver are disposed on a second surface that opposes the first surface. The optical emitter and/or the optical receiver may be disposed on a surface of a printed circuit board.

The autoinjector according to the first aspect of the invention may, additionally or alternatively, include a first sensor disposed between a proximal end and a distal end of the housing, where the first sensor is configured to detect a first electrical signal generated by applying an insertion force to insert the needle into a user. The first sensor may include a force sensor.

The autoinjector according to the first aspect of the invention may, additionally or alternatively, include a second sensor disposed between a proximal end and a distal end of the housing, where the second sensor is configured to detect an ambient temperature in proximity to the container. The second sensor may include a thermistor.

According to the first aspect of the invention, the housing may include an optically shielded housing, and/or the housing may include an optically blocking label.

In a second aspect of the invention, the present disclosure encompasses an autoinjector includes a housing; a needle arranged at a distal end of the housing; a container disposed within the housing and configured to contain medicament; a plunger slidably disposed within the container; a plunger rod configured to push the plunger through the container to dispense the medicament through the needle when the container contains medicament, where the plunger rod includes one or more optical transmission regions; a mechanism configured so that a dispensing movement of the plunger rod causes the mechanism to generate a first optical signal and transmit the first optical signal through at least one of said one or more optical transmission regions; and an optical sensor configured to detect said first optical signal generated by the mechanism.

In a third aspect of the invention, the present disclosure encompasses an autoinjector includes a housing; a needle arranged at a distal end of the housing; a needle guard configured to expose the needle when an insertion force is applied to insert the needle into a user; a container disposed within the housing and configured to contain medicament; a plunger slidably disposed within the container; a plunger rod configured to push the plunger through the container to dispense the medicament through the needle when the container contains medicament and when the needle is exposed, where the plunger rod includes one or more optical transmission regions; an optical mechanism configured to emit a first optical signal through at least one of the one or more optical transmission regions of the plunger rod and to receive the first optical signal, where a dispensing movement of the plunger rod enables transmission of the first optical signal; and an electrical mechanism configured to contact a portion of the needle guard, where a compression movement of the needle guard generates a first electrical signal.

In a fourth aspect of the invention, the present disclosure encompasses a system including an autoinjector (e.g., any described herein, such as those of the first, second or third aspects of the invention) and a processor configured to process one or more optical signals to track the dispensing movement of the plunger rod.

In a fifth aspect of the invention, the present disclosure encompasses a method of detecting a dispensing movement of a plunger rod within an autoinjector including detecting one or more optical signals of an optical emitter as the plunger rod moves during the dispensing movement, where the plunger rod includes one or more optical transmission regions, and where the dispensing movement of the plunger rod provides optical communication between the optical emitter and one or more transmission regions to transmit the one or more optical signals.

In a sixth aspect of the invention, the present disclosure encompasses a method of detecting a dispensing movement of a plunger rod within an autoinjector including detecting an optical signal generated due to movement of the plunger rod during the dispensing movement.

According to the fifth or sixth aspect of the invention, the plunger rod may be configured to transmit a plurality of optical signals during the dispensing movement of the plunger rod. Optionally, the plunger rod may include a plurality of optical transmission regions. Further optionally, a first optical transmission region of the plurality of optical transmission regions may be configured to indicate dose start of a medicament, and/or a last optical transmission region of the plurality of optical transmission regions may be configured to indicate dose end of the medicament. Additionally or alternatively, each optical transmission region of the plurality of optical transmission regions may be configured to transmit a corresponding optical signal during the dispensing movement of the plunger rod.

According to the fifth or sixth aspect of the invention, the autoinjector may include an audible clicker configured to produce one or more audible clicks during the dispensing movement of the plunger rod. The audible clicker may include a ring surrounding the plunger rod, and the ring includes a deflectable protrusion configured to contact the plunger rod and produce the one or more audible clicks. Optionally, the plunger rod may include a ridged surface configured to contact the deflectable protrusion of the audible clicker. Further optionally, each ridge of the ridged surface may be configured to cause the deflectable protrusion to deflect during the dispensing movement of the plunger rod.

According to a fifth or sixth aspect of the invention, a first sensor may be disposed between a proximal end and a distal end of a housing of the autoinjector. The first sensor may include a voltage sensor or a force sensor. Additionally or alternatively, the first sensor may be disposed on a surface of a printed circuit board. Additionally or alternatively, the first sensor may be further configured to detect a first electrical signal generated by applying an insertion force to insert a needle into a user.

According to the fifth or sixth aspects of the invention, a second sensor may be disposed between a proximal end and a distal end of the housing, and the second sensor is configured to detect an ambient temperature in proximity to a container configured to contain a medicament. The second sensor may include a thermistor.

The fifth or sixth aspects of the invention may include sending, via a wireless transfer protocol, signals from a first sensor, if present, and a second sensor, if present, to a mobile device. Optionally, the fifth or sixth aspects of the invention may further include displaying information about the dispensing movement on the mobile device.

The fifth or sixth aspects of the invention may include measuring a temperature of medicament within the autoinjector or an ambient temperature within the autoinjector using a temperature sensor. Optionally, the fifth or sixth aspects of the invention may include sending, via a wireless transfer protocol module, signals from the temperature sensor to a mobile device. Additionally or alternatively, the fifth or sixth aspects of the invention may include displaying the temperature of the medicament or the ambient temperature on a mobile device; indicating that the temperature of the medicament or the ambient temperature is above a threshold temperature for use of the autoinjector; or both.

According to a seventh aspect of the invention, also provided is a composition comprising lenacapavir or a pharmaceutically accepted salt thereof for use in the prevention or treatment of HIV, where the composition is administered via an autoinjector (e.g., any described herein, such as those of the first, second or third aspects of the invention). The administration may be subcutaneous or intramuscular.

According to an eighth aspect of the invention, also provided is the use of lenacapavir or a pharmaceutically accepted salt thereof for the manufacture of a medicament for the prevention or treatment of HIV, wherein the prevention or treatment comprises administering the medicament via an autoinjector (e.g., any described herein, such as those of the first, second or third aspects of the invention). The administration may be subcutaneous or intramuscular.

The details of one or more embodiments of the subject matter described herein are set forth in the accompanying drawings and the description below. Other features, objects, and advantages of the subject matter will be apparent from the description and drawings, and from the claims.

### DESCRIPTION OF DRAWINGS

FIG. 1 illustrates an example of an autoinjector.

FIG. 2A is a cut-away view of an example autoinjector.

FIG. 2B is a cross-section view of the autoinjector of FIG. 2A.

FIG. 2C is another cut-away view of the autoinjector of FIG. 2A.

FIG. 3 is a cross-section view of another example autoinjector.

FIG. 4A is a perspective view of a plunger rod of the autoinjector of FIG. 3.

FIG. 4B is a perspective view of a needle guard of the autoinjector of FIG. 3

FIG. 4C is a perspective view of a syringe carrier of the autoinjector of FIG. 3.

FIG. 4D is a cross-section view of the syringe carrier of FIG. 4C.

FIG. 5 is a perspective view of another example autoinjector.

FIGS. 6A-6B illustrate an example autoinjector during operation of the autoinjector.

FIG. 7 illustrates a process of dispensing a dose of medicament using an example autoinjector.

FIG. 8 is a flow chart for an example method of dispensing a dose of medicament using an autoinjector.

FIG. 9 is an example plot of temperature readings of a medicament using an example autoinjector.

FIG. 10 is an example plot of the performance of an example infrared detector in various lighting conditions.

Like reference numbers and designations in the various drawings indicate like elements.

### DETAILED DESCRIPTION

The present systems, devices, and methods can sense a dispensing movement of a plunger rod within an autoinjector. For example, one or more optical transmission regions on the plunger rod of the autoinjector can enable optical signals to be transmitted and received by a sensing assembly to track the dispensing movement of the plunger rod. The signals can include voltage or other electrical measurements that indicate that an optical transmission region has reached a certain location within the autoinjector or that a certain number of optical transmission regions has progressed through the autoinjector. The devices can include a sensing assembly that can determine that the plunger rod has completed the dispensing movement after receiving a predetermined number of signals. The sensing assembly can also track the dispensing movement of the plunger rod. Tracking the dispensing movement of the plunger rod can be advantageous because the dispensing movement of the plunger rod corresponds to the amount of medicament delivered to the subject (e.g., a patient). If the plunger rod does not complete the dispensing movement, then the full dose of medicament is not injected into the patient. The sensing assembly can be used to determine that the full dose is injected into the patient by tracking the dispensing movement. Tracking movement of the plunger rod throughout the dispensing movement can be advantageous for determining how much of a medicament has been delivered and whether the amount of medicament injected exceeds a minimum dose volume threshold for medicament efficacy.

The present systems, devices, and methods can also sense whether a needle of the autoinjector is inserted into a patient to a sufficient depth and/or whether the insertion depth is maintained during delivery of the medicament. For example, an electrical contact between a needle guard and a particular location in the autoinjector can determine whether the needle has been inserted to a sufficient depth. Determining that the needle is sufficiently inserted into the patient can be advantageous because if the needle is not sufficiently inserted into the patient, the patient may not receive the medicament properly.

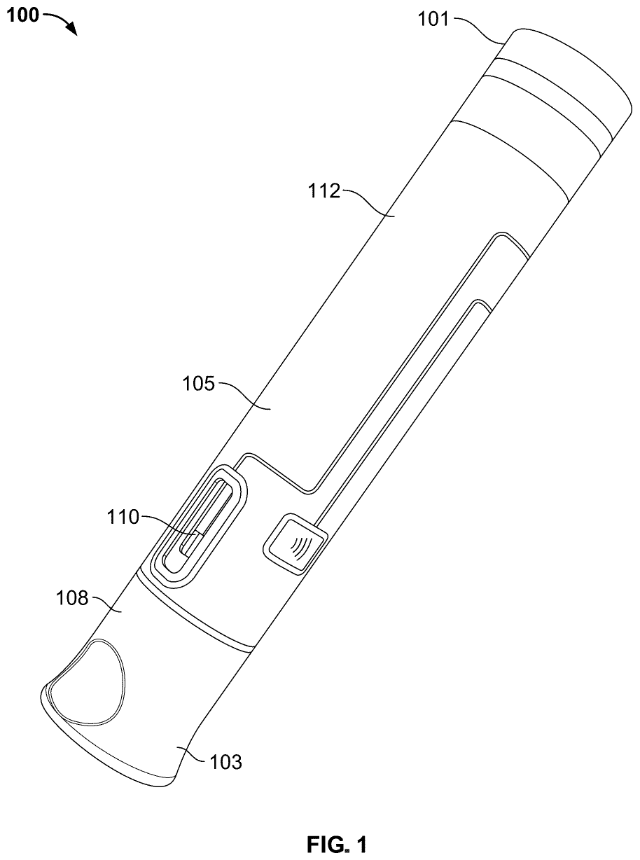

FIG. 1 illustrates an autoinjector 100 that can sense a dispensing movement of a plunger rod within the autoinjector 100 and can sense whether a needle of the autoinjector 100 is inserted into a patient to a sufficient depth. Alternatively, or in addition, the autoinjector 100 can sense whether an insertion depth of a needle is maintained during dispensing or delivery. The autoinjector 100 includes a housing 105, a proximal end 101 of the housing 105, and a front cap 108 attached to a distal end 103 of the housing 105. The front cap 108 covers a needle assembly configured to be inserted into the user during injection. The user removes the front cap 108 prior to use of the autoinjector 100. A sensing assembly can be located somewhere between the proximal end 101 and the distal end 103 of the housing 105. The sensing assembly can sense a dispensing movement of a plunger rod within the autoinjector 100, e.g., to determine that the plunger rod has completed the dispensing movement, as discussed below. The housing 105 can include flanges configured to accommodate the user's fingers.

The housing 105 can include an optional label 112 that provides information about the autoinjector 100. For example, the label 112 can include medicament information, such as the type of medicament, the size of the dose, and the delivery time of the dose. Optionally, the housing 105 does not include a label 112.

The housing 105 also includes an optional window 110, through which a user can see medicament contained within the autoinjector 100, e.g., within a container of the autoinjector 100 (see discussion below). The window 110 may help a user determine whether the autoinjector 100 has been used. Before use of the autoinjector 100, the user can see through the window 110 to determine whether there is medicament within the autoinjector 100, for example, to determine that the autoinjector has not been used. During use of the autoinjector 100, the user may look through the window 110 to determine whether the volume of medicament in the autoinjector 100 is decreasing. After use of the autoinjector 100, the user may look through the window 110 to determine that there is no medicament in the autoinjector 100, for example, to determine that the autoinjector 100 has been used.

Different injection sites, patient age and patient body mass may affect the recommended needle length, and higher viscosity drugs will require a larger diameter needle to prevent the injection force becoming too high for the device. Advantageously, selecting needle gauge based on viscosity of drug being administered can ensure that the full dose of drug is administered without undue strain. Typically, injection force is less than 40 Newtons through needle gauge selection. Preferably, injection force is less than 20 Newtons through needle gauge selection.

Needle gauges disclosed herein are provided in Birmingham Wire Gauge (also known as: Birmingham Gauge or Stubs Iron Wire Gauge), abbreviated as “gauge” or G. In accordance with ISO standard ISO 9626:2016, needle wall thickness designations include Regular Wall, Thin Wall, Extra Thin Wall, and Ultra Thin Wall. Regular Wall thickness is abbreviated to RW. Thin Wall thickness is abbreviated to TW. Extra Thin Wall thickness is abbreviated to ETW. Ultra Thin Wall is abbreviated to UTW. Alternatively, needle wall thickness may be Special Thin Wall; Special Thin Wall thickness is abbreviated as STW. Viscosity is provided in centipoise (cP), where one centipoise is equivalent to one millipascal-second.

The autoinjector 100 may be used for subcutaneous injections, which are directed into fat tissue between the skin and the muscle of the patient. Subcutaneous injections typically involve shorter and narrower needles than intramuscular injections, which are directed into the muscle of a patient. Needles for subcutaneous injections are typically 34-27 gauge and 4-12 mm in insertion depth (needle extension) for subcutaneous injections into the abdomen. Insertion depth for a subcutaneous injection may be 4-8 mm. For subcutaneous injection, needle length may be 8-13 mm. For delivery of 2.25-3 mL dose of liquid (medicament) with a viscosity of up to 5 cP using a needle with a needle length 8-13 mm, needle gauge may be 29G RW or TW, or 27G RW. For delivery of 2.25-3 mL dose of liquid (medicament) with a viscosity of up to 30 cP using a needle with a needle length 8-13 mm, needle gauge may be 27G TW or 25G RW. For delivery of 2.25-3 mL dose of liquid (medicament) with a viscosity of up to 50 cP using a needle with a needle length 8-13 mm, needle gauge may be 25G TW. For delivery of 2.25-3 mL dose of liquid (medicament) with a viscosity of up to 160 cP using a needle with a needle length 8-13 mm, needle gauge may be 25G STW or 23G RW. For delivery of 2.25-3 mL dose of liquid (medicament) with a viscosity of up to 300 cP using a needle with a needle length 8-13 mm, needle gauge may be 22G ETW. For delivery of 2.25-3 mL dose of liquid (medicament) with a viscosity of up to 600 cP using a needle with a needle length 8-13 mm, needle gauge may be 18G ETW or 18G UTW. For delivery of 1 mL dose of liquid (medicament) with a viscosity of up to 10 cP using a needle with a needle length 8-13 mm, needle gauge may be 29G RW or TW, or 27G RW. For delivery of 1 mL dose of liquid (medicament) with a viscosity of up to 30 cP using a needle with a needle length 8-13 mm, needle gauge may be 27G TW or 25G RW. For delivery of 1 mL dose of liquid (medicament) with a viscosity of up to 80 cP using a needle with a needle length 8-13 mm, needle gauge may be 25G TW. For delivery of 1 mL dose of liquid (medicament) with a viscosity of up to 350 cP using a needle with a needle length 8-13 mm, needle gauge may be 25G STW or 23G RW. For delivery of 1 mL dose of liquid (medicament) with a viscosity of up to 600 cP using a needle with a needle length 8-13 mm, needle gauge may be 18-22G ETW.

The autoinjector 100 may be used for intramuscular injections. Needles for intramuscular injections on adults are typically 25-20 gauge and 15-25 mm in insertion depth (needle extension). Alternatively, insertion depth for an intramuscular injection may be 25-50 mm. For intramuscular injection, needle length may be 1-1.5 inches (25.4-38.1 mm). For delivery of 2.25-3 mL dose of liquid (medicament) with a viscosity of up to 1 cP using a needle with a needle length 25.4-38.1 mm, needle gauge may be 29G RW or TW, or 27G RW. For delivery of 2.25-3 mL dose of liquid (medicament) with a viscosity of up to 5 cP using a needle with a needle length 25.4-38.1 mm, needle gauge may be 27G TW or 25G RW. For delivery of 2.25-3 mL dose of liquid (medicament) with a viscosity of up to 10 cP using a needle with a needle length 25.4-38.1 mm, needle gauge may be 25G TW. For delivery of 2.25-3 mL dose of liquid (medicament) with a viscosity of up to 40 cP using a needle with a needle length 25.4-38.1 mm, needle gauge may be 25G STW or 23G RW. For delivery of 2.25-3 mL dose of liquid (medicament) with a viscosity of up to 200 cP using a needle with a needle length 25.4-38.1 mm, needle gauge may be 22G ETW. For delivery of 2.25-3 mL dose of liquid (medicament) with a viscosity of up to 600 cP using a needle with a needle length 25.4-38.1 mm, needle gauge may be 18G ETW. For delivery of 1 mL dose of liquid (medicament) with a viscosity of up to 5 cP using a needle with a needle length 25.4-38.1 mm, needle gauge may be 29G RW or TW, or 27G RW. For delivery of 1 mL dose of liquid (medicament) with a viscosity of up to 10 cP using a needle with a needle length 25.4-38.1 mm, needle gauge may be 27G TW or 25G RW. For delivery of 1 mL dose of liquid (medicament) with a viscosity of up to 30 cP using a needle with a needle length 25.4-38.1 mm, needle gauge may be 25G TW. For delivery of 1 mL dose of liquid (medicament) with a viscosity of up to 50 cP using a needle with a needle length 25.4-38.1 mm, needle gauge may be 25G STW or 23G RW. For delivery of 1 mL dose of liquid (medicament) with a viscosity of greater than 50 cP using a needle with a needle length 25.4-38.1 mm, needle gauge may be 18-22G ETW.

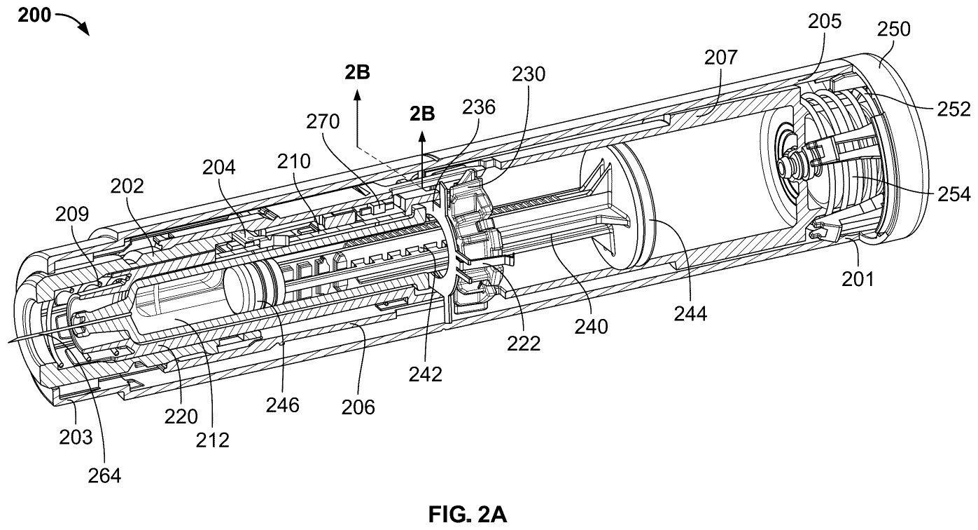

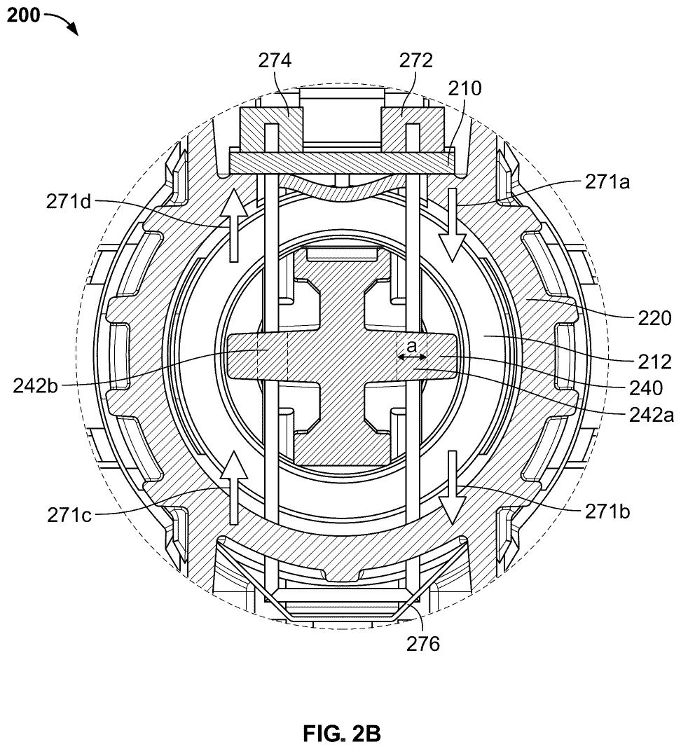

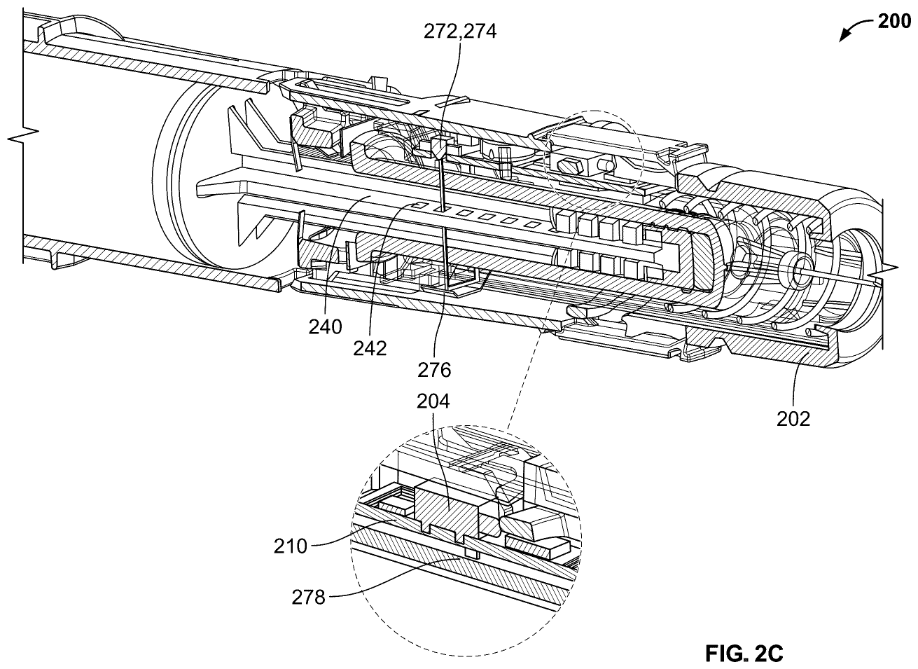

FIG. 2A is a cut-away view of an example autoinjector 200. The autoinjector 200 includes a housing 205. A needle 264 is disposed at a distal end 203 of the housing 205. A needle guard 202 protects the needle 264 when the autoinjector is not in use. The autoinjector 200 includes a container 212 (e.g., a syringe) within the housing 205 for holding a medicament to be injected. The container 212 may have an internal volume of 1.5-3 mL. The container 212 may have an internal volume of 1 mL, 2.25 mL, 3 mL, or 5 mL. The medicament may have a volume of 0.5-5 mL. Preferably the medicament has a volume of 1.5-3 mL. For example, the medicament may have a volume of 1.5 mL, 2.25 mL or 3 mL. The container 212 is disposed in a syringe carrier 220. The syringe carrier 220 and a syringe backstop 222 hold and maintain the position of the container 212 within the housing 205. A plunger 246 is slidably disposed within the container 212. A plunger rod 240 is configured to push the plunger 246 through the container 212 to dispense the medicament through the needle 264. The plunger rod 240 protrudes through a lock ring 230. The plunger rod 240 and lock ring 230 are disposed within a delivery chamber 207.

At a proximal end 201 of the autoinjector 200, a rear case 250 is coupled to the housing 205. The proximal end 201 also includes a spring 252 (e.g., an anti-rattle spring) and a gas canister assembly 254. The spring 252 is disposed between the rear case 250 and the delivery chamber 207. The spring 252 biases the delivery chamber 207 toward the distal end 203. The gas canister assembly 254 includes pressurized gas that when released provides a force acting on the plunger rod 240 to push the plunger 246 through the container 212 in a dispensing movement to dispense the medicament. During a dispensing movement, the pressurized gas is contained in the delivery chamber 207, and a piston seal 244 reduces leakage around the plunger rod 240.

Activation of the gas canister assembly 254 can occur by a user fully depressing the needle guard 202 into the housing 205 (e.g., by pressing the needle guard 202 and the autoinjector 200 against his or her skin), such that the needle guard 202 moves the transfer sleeve 206 proximally. In turn, this causes all of the internal components (e.g., including the delivery chamber 207) to move a relatively small proximal distance against the bias of the spring 252 disposed at the proximal end 201 of the autoinjector 200. For example, the relatively small proximal distance can be a distance of 1-10 millimeters. Upon moving such a relatively small proximal distance, the gas canister assembly 254 can be activated (e.g., by movement of a firing pin that pierces an end of a gas canister within the gas canister assembly 254), thereby releasing compressed gas from within. The compressed gas can include, for example, argon, carbon dioxide, krypton, xenon, etc. Optionally, depression of the needle guard 202 can cause an internal component to contact a sensing assembly including a force sensor, such that the force sensor can measure the insertion force of the needle into the user (e.g., as described herein).

The needle guard 202 includes a needle guard contact 204. The needle guard contact 204 can be coupled to a crossbar of the needle guard 202. For example, the needle guard contact 204 can be heat staked to the crossbar. The needle guard contact 204 can be formed of a single stamped metallic piece with one or more flexible arms. The needle guard contact 204 is configured to contact a conductive portion of the main PCB 210 (e.g., one or more contact regions that are plated on the edge of the main PCB 210) when the needle guard 202 is in a retracted position. The contact between the needle guard contact 204 and the main PCB 210 can be detected by a second sensor (e.g., a voltage sensor, resistance sensor, or other sensor configured to detect an electrical signal as the needle guard moves). Contact between the needle guard contact 204 and the main PCB 210 indicates that the needle 264 of the autoinjector 200 is exposed, and the autoinjector 200 is ready for the dosing process to begin. The location and configuration of the needle guard contact 204 can be optimized to balance exposure of the needle, insertion depth of the needle into the patient's skin, and/or dosing initiation.

A needle guard spring 209 is disposed between the needle guard 202 and the syringe carrier 220. The needle guard spring 209 biases the needle guard 202 toward the distal end 203 of the housing 205, causing the needle guard 202 to extend from the housing 205 and cover the needle 264. Retraction of the needle guard 202 compresses the needle guard spring 209. The needle guard 202 can be retracted by exerting a force on the distal end 203. Contact between the needle guard contact 204 and the main PCB 210 can indicate that a sufficient force has been applied to the autoinjector 200 to retract the needle guard 202 and expose the needle 264. Upon removing the sufficient force applied to the autoinjector 200 (e.g., after the dose is dispensed), the needle guard 202 can then extend from the housing 205 to cover the needle 264.

The lock ring 230 includes lock ring contact 236. The lock ring contact 236 can be, for example, a sheet metal plate with one or more deflectable arms. The lock ring contact 236 can be positioned on the lock ring 230 using one or more posts protruding from the lock ring 230. The lock ring contact 236 can include an opening to accommodate passage of the plunger rod 240 during a dispensing movement. When a dispensing movement is initiated, the lock ring 230 shunts toward the syringe carrier 220 with the plunger rod 240. Electrical contact between the lock ring contact 236 and the main PCB 210 indicates that the dispensing movement has started. Additionally, hard stops on the lock ring 230 stop the lock ring 230 from travelling too far within the housing 205.

The sensing assembly of the autoinjector 200 includes a main PCB 210 that includes electronics to sense optical transmissions, process data, and/or communicate with an external device (e.g., a mobile device, smartphone, or tablet) through a wireless communication protocol (e.g., short range radio communication, near field communication, wireless transfer protocol, Wi-Fi). The main PCB 210 is coupled to the syringe carrier 220. For example, the main PCB 210 can be heat staked to the syringe carrier 220. The transfer sleeve 206 can slide between the main PCB 210 and the housing 205.

The main PCB 210 includes an optical detection unit 270 to detect dosing progress of the autoinjector 200. The plunger rod 240 includes one or more apertures 242 through which an optical signal can be transmitted to the optical detection unit 270. When multiple apertures 242 are included on the plunger rod 240, the optical detection unit 270 may generate a signal corresponding to the passage of each aperture 242. The main PCB 210 can determine that the dose has been completed based on the number of signals generated by the optical detection unit 270 and the number of apertures 242 on the plunger rod 240.

FIG. 2B is a cross-section view of the autoinjector 200 showing the optical path for an optical signal used to detect the dose progress of the autoinjector 200. The main PCB 210 includes an optical emitter 272 and an optical receiver 274. On the opposite side of the syringe carrier 220 from the main PCB 210, the syringe carrier 220 includes an optical reflector 276 configured to reflect an optical signal from the optical emitter 272 to the optical receiver 274.

During a dispensing movement, the plunger rod 240 translates in the container 212. When a first aperture 242a of the plunger rod 240 aligns with the optical path, light is transmitted 271a from the optical emitter 272 through the first aperture 242a to the optical reflector 276. The light reflects 271b from a first portion of the optical reflector 276 to a second portion of the optical reflector 276. The light reflects 271c from the second portion of the optical reflector 276 and transmits 271d through a second aperture 242b and to the optical receiver 274. The light emitted by the optical emitter 272 can be, for example, visible light (e.g., 400-700 nm wavelength) or infrared light (e.g., 700 nm-1 mm wavelength). The first and second apertures 242a,b can be arranged and aligned in any useful manner within the plunger rod 240 in order to provide any continuous optical path between the optical transmitter 272, optical reflector 276, and optical receiver 274. The position of the optical transmitter 272, the optical receiver 274, and the optical reflector 276 can be adjusted to account for refraction of the light through the container. Alternatively, or additionally, the local geometry of the container 212 in the optical path 271 can be adjusted to mitigate refraction of light in the optical path 271.

FIG. 2C is a partial cut-away view of the autoinjector 200. As shown, an aperture 242 of the plunger rod 240 is aligned with the optical emitter 272 and the optical receiver 274 enabling transmission of light from the optical emitter 272 through the aperture 242 to the optical reflector 276 and from the optical reflector 276 through the aperture 242 to the optical receiver 274. The main PCB 210 also includes a temperature sensor 278 to measure a temperature of medicament in the container 212 and/or the temperature of the ambient environment within the housing near the container 212. In some implementations, the temperature sensor 278 is included as a part of a short range wireless communications module.

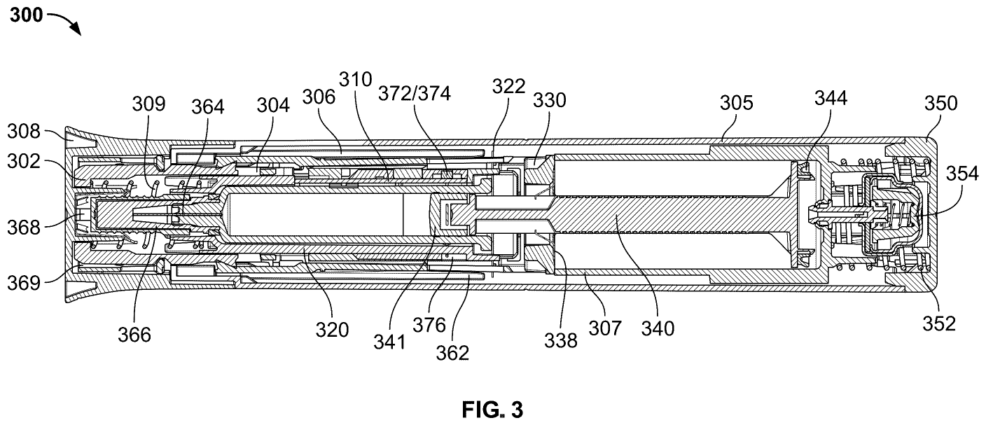

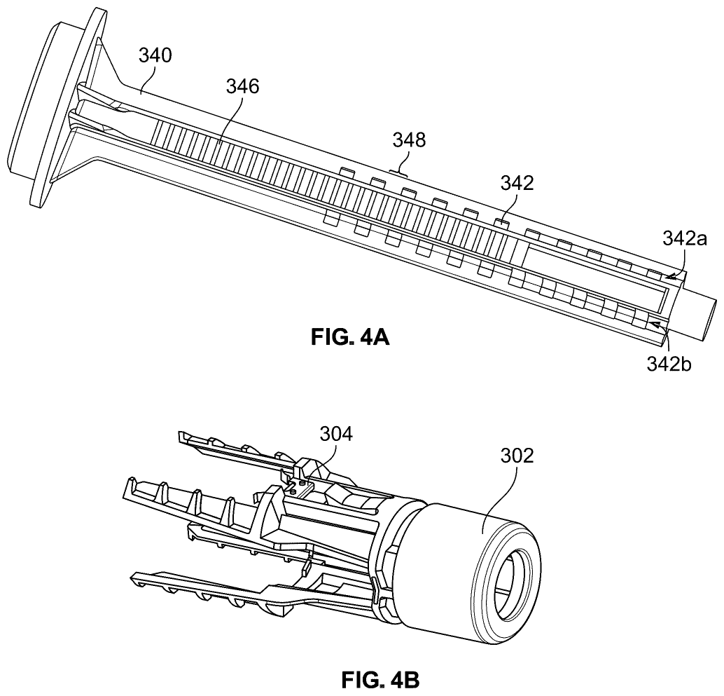

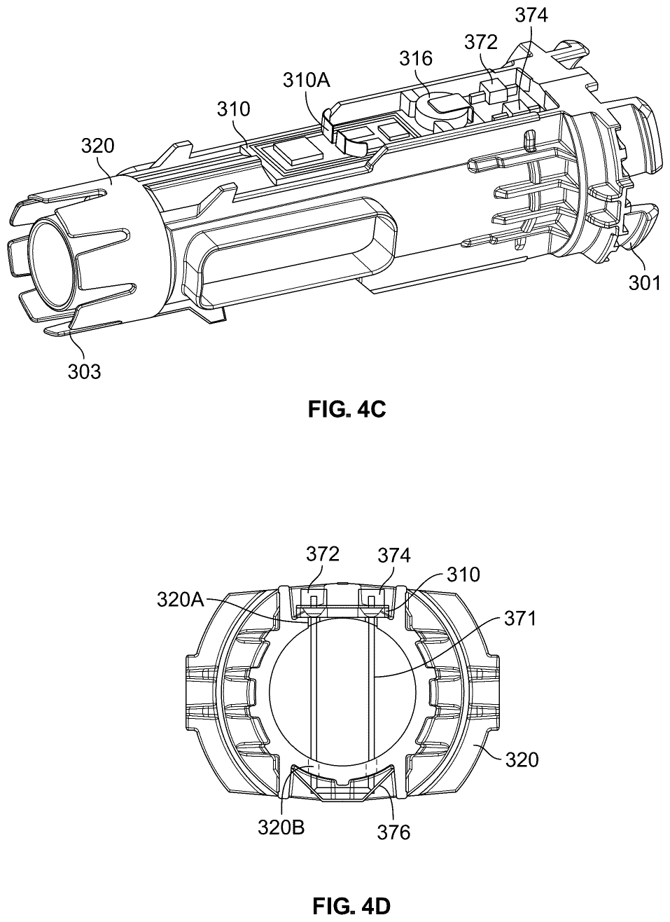

FIG. 3 is a cross-section view of yet another example autoinjector 300. FIGS. 4A-4D illustrate example components of the autoinjector 300. The autoinjector 300 includes a housing 305 and front cap 308 coupled to a distal end of the housing 305. The front cap 308 includes a needle shield remover 368 that can remove a needle shield 366 from a needle 364 when the front cap 308 is removed from the housing 305. The front cap 308 also includes an anti-drop ring 369 to reduce the likelihood of accidental removal of the front cap 308 due to the autoinjector 300 being dropped by a user.

The needle 364 is connected to container 362. The needle shield 366 and needle guard 302 protect the needle 364 prior to use of the autoinjector 300. The needle guard 302 is biased away from the container 362 by the needle guard spring 309. The needle guard 302 includes a needle guard contact 304 that can contact the main PCB contact 310 during use of the autoinjector 300 to indicate that the needle 364 is inserted to an appropriate depth in a subject.

The container 362 can include a medicament to be injected into a patient. The container 362 may have an internal volume of 1.5-3 mL. The container 362 may have an internal volume of 1 mL, 2.25 mL, 3 mL, or 5 mL. The medicament may have a volume of 0.5-5 mL. Preferably the medicament has a volume of 1.5-3 mL. For example, the medicament may have a volume of 1.5 mL, 2.25 mL or 3 mL The container 362 is held in place within the autoinjector 300 by syringe carrier 320. A syringe backstop 322 holds the syringe carrier 320 in place restricting movement of the syringe carrier 320 toward the rear case 350.

A transfer sleeve 306 is positioned around the syringe carrier 320. The transfer sleeve 306 interfaces between the needle guard 302 and the delivery chamber 307. The main PCB 310 is located between the transfer sleeve 306 and the syringe carrier 320. Alternatively, the main PCB may optionally be located between the transfer sleeve 306 and the housing 305 in a fixed location relative to the housing 305.

The delivery chamber 307 is disposed between the lock ring 330 and the gas canister assembly 354. The delivery chamber 307 includes the plunger rod 340. The piston seal 344 forms a seal between the plunger rod 340 and sides of the delivery chamber 307 to reduce leakage of the pressurized gas from the gas canister assembly 354 past the plunger rod 340. The gas canister assembly 354 is disposed between the delivery chamber 307 and the rear case 350. A spring 352 is disposed around the gas canister assembly 354. The spring 352 biases the delivery chamber 307 toward the needle 364.

The plunger rod 340 protrudes through a lock ring 330 to exert a force on the plunger 341 that is slidably disposed within the container 362 to dispense the contents of the container 362. A lock ring contact is disposed between the lock ring 330 and syringe carrier 320 on a distal side of the lock ring 330, and a circular clicker 338 (e.g., a ring configured to include an opening therein) is disposed on a proximal side of the lock ring 330.

As seen in FIG. 4A, the plunger rod 340 includes optical transmission regions (e.g., aperture 342) disposed along sides of the plunger rod 340. The apertures 342 are spaced apart from one another with a spacing 348 between successive apertures 342. A plunger rod 340 may include a first series 342a of apertures and a second series 342b of apertures, in which the first and second series 342a,b extend along the longitudinal axis of the plunger rod 340, and in which the first series 342a is located on a first lateral edge of the plunger rod 340 and the second series 342b is located on a second lateral edge of the plunger rod 340. The first and second lateral edges may be approximately parallel to one another (e.g., and approximately parallel to the longitudinal axis of the plunger rod 340).

The apertures 342 enable an optical signal to be transmitted from the optical emitter 372 to the optical receiver 374 to track the progress of the dose. For example, an optical signal can be received corresponding to the passage of an aperture 342 through the optical path 371. The progress of the dose can be tracked based on a number of optical signals received during the dispensing movement. In some implementations, the optical transmission regions include optically transparent material (e.g., infrared transparent material, glass, etc.).

The plunger rod 340 also includes ridges 346. The ridges 346 can contact the circular clicker 338 during a dispensing movement to emit one or more audible clicks as the dose progresses. The circular clicker 338 interfaces with the plunger rod 340. Openings and other structural elements (e.g., contacts, stops, arms, etc.) for the lock ring 330 and the circular clicker 338 can be configured to minimize impeding a pathway for the dispensing movement of the plunger rod 340. As the plunger rod 340 moves through the circular clicker 338 during a dispensing movement, the circular clicker 338 emits one or more audible click sounds that can be used to track the dispensing movement. For example, the circular clicker 338 can have a deflectable protrusion configured to contact a surface of the plunger rod 340 (e.g., ridges 346). The dispensing movement of the plunger rod 340 can deflect the deflectable protrusion to cause the circular clicker 338 to produce one or more audible clicks, vibrations or the like.

The circular clicker 338 can include multiple deflectable protrusions. Where the circular clicker 338 includes multiple deflectable protrusions, the plunger rod 340 can include multiple surfaces configured to deflect the deflectable protrusions. For example, and without limitation, the plunger rod 340 can include multiple surfaces configured to deflect the deflectable protrusions, in which each surface can in turn include a plurality of ridges, bumps, projections, or other non-continuous structures along the surface to provide a ridged surface or other non-continuous surface (see discussion below). Alternatively, the circular clicker 338 may include a single deflectable protrusion. Where the circular clicker 338 includes a single deflectable protrusion, the plunger rod 340 can include a single surface (e.g., ridges 346) configured to deflect the deflectable protrusion. Any number of deflectable protrusions can be employed to produce one or more clicks (e.g., a single deflectable protrusion or multiple deflectable protrusions). In one non-limiting example, a single deflectable protrusion can be employed to reduce the possibility of interference between multiple audible clicks. In another non-limiting example, multiple deflectable protrusions can be employed to operate in sync to provide a more distinct click sound, in which interference can be minimized, e.g., by control of tolerancing and/or alignment.

The circular clicker 338 can be a portion of the autoinjector housing. For example, the autoinjector housing can include a deflectable protrusion that contacts the plunger rod 340. The plunger rod 340 can include one or more deflectable protrusions, and the housing (or another internal component of the autoinjector) includes a surface configured to deflect the one or more deflectable protrusions.

The ridged surface 346 can include any appropriate number (e.g., a plurality, such as 1-100, 1-90, 1-80, 1-70, 1-60, 1-50, 1-40, 1-30, 1-20, 2-100, 2-50, 3-100, 3-50, 4-100, 4-50, 5-100, 5-50, 5-15, 8-100, 8-50, 10-100, 10-50, 10) of ridges. Each ridge of a ridged surface can deflect the deflectable protrusion to cause the deflectable protrusion to produce a signal (e.g., an audible click, a vibration, etc.) that can be sensed by a sensing assembly. For example, an example sensing output can include multiple signals, in which each signal corresponds to a ridge of a ridged surface. In turn, the number of signals can be used to track the number of ridges along the plunger rod 340 that passed by the circular clicker 338 during a dispensing movement of the plunger rod 340. For example, a sensing assembly can use the received signals to determine that the plunger rod 340 has completed the dispensing movement after a predetermined number (e.g., a plurality, such as 1-100, 1-90, 1-80, 1-70, 1-60, 1-50, 1-40, 1-30, 1-20, 2-100, 2-50, 3-100, 3-50, 4-100, 4-50, 5-100, 5-50, 5-15, 8-100, 8-50, 10-100, 10-50, 10) of signals. The ridged surface can be any non-continuous surface configured to provide a plurality of non-continuous structures (e.g., bumps, projections, ribs, and the like upon that surface).

As seen in FIGS. 4C-4D, the syringe carrier 320 includes a distal end 303 that interfaces with the needle guard 302 and the needle guard spring 309. The proximal end 301 of the syringe carrier 320 interfaces with the syringe backstop 322. The main PCB 310 is disposed on an outer surface of the syringe carrier 320. The main PCB 310 includes a battery 316 (e.g., a coin battery). The main PCB 310 also includes main PCB contact 310A that can be contacted by the needle guard contact 304 when the needle guard 302 is in a retracted position.

The main PCB 310 also includes an optical emitter 372 and optical receiver 374. An optical path 371 is formed in the syringe carrier through openings 320A and 320B. The syringe carrier 320 also includes an optical reflector 376 on a side opposite the main PCB 310. Light emitted from the optical emitter 372 passes through openings 320A and 320B along optical path 371 to reach the optical reflector 376. The light is then reflected from the optical reflector 376 and passes through openings 320B and 320A to reach the optical receiver 374.

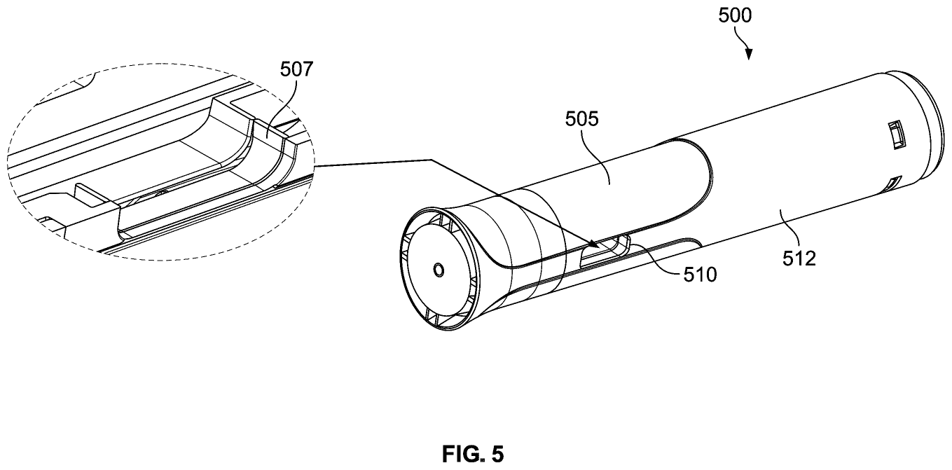

FIG. 5 illustrates an example autoinjector 500 that includes optically shielded components. The autoinjector 500 includes an optically shielded housing 505 that reduces an amount of light that can propagate into the interior of the housing 505 as compared with a non-optically shielded housing. The optically shielded housing 505 can include, for example, polymer additives (e.g., titanium dioxide TiO₂) that increase the opacity of the housing 505 for desired wavelengths of light. The autoinjector 500 can also include an optically blocking label 512 that includes, for example, a metallized portion and/or light blocking inks and films to block a desired wavelength of light. An optical baffle 507 can be included around window 510 to limit the exposure of internal components of the autoinjector 500 to light allowed into the interior of the autoinjector 500 through the window 510.

Optical shielding can be important to maintain consistent performance of an optical sensor in a variety of lighting scenarios. For example, in dark conditions, an optical sensor may detect small amounts of light from ambient light sources improving detection of optical signals from an optical emitter. In a bright environment (e.g., a sunny, outdoor environment), without optical shielding, the optical sensor may be nearly saturated by ambient light causing a small signal to noise ratio resulting in decreased detection of optical signals from an optical emitter. With optical shielding, the ambient light reaching the optical sensor can be decreased improving detection of optical signals from an optical emitter.

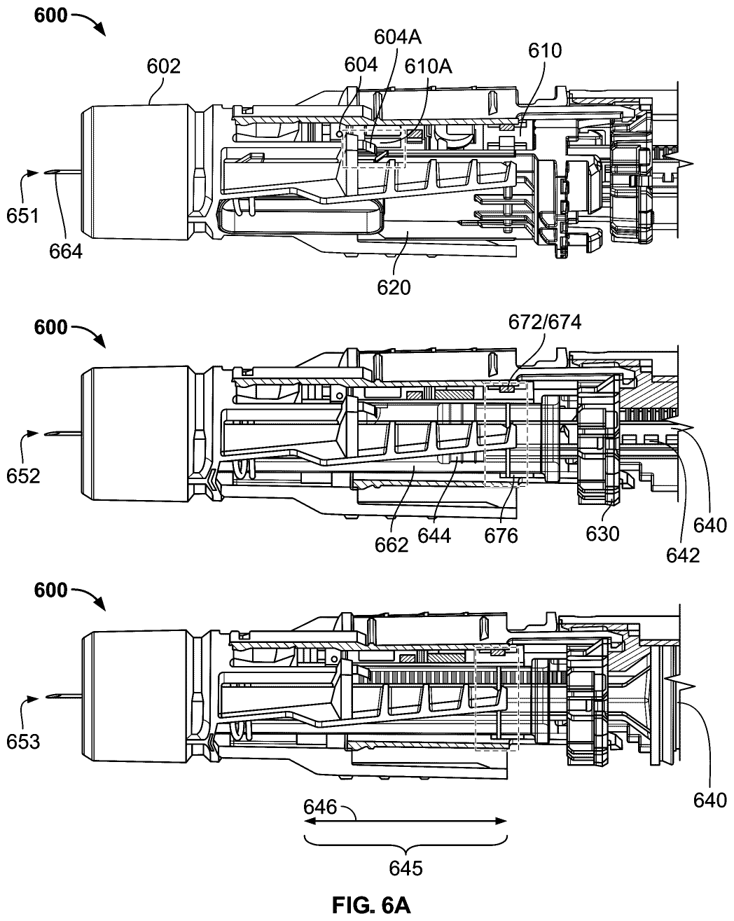

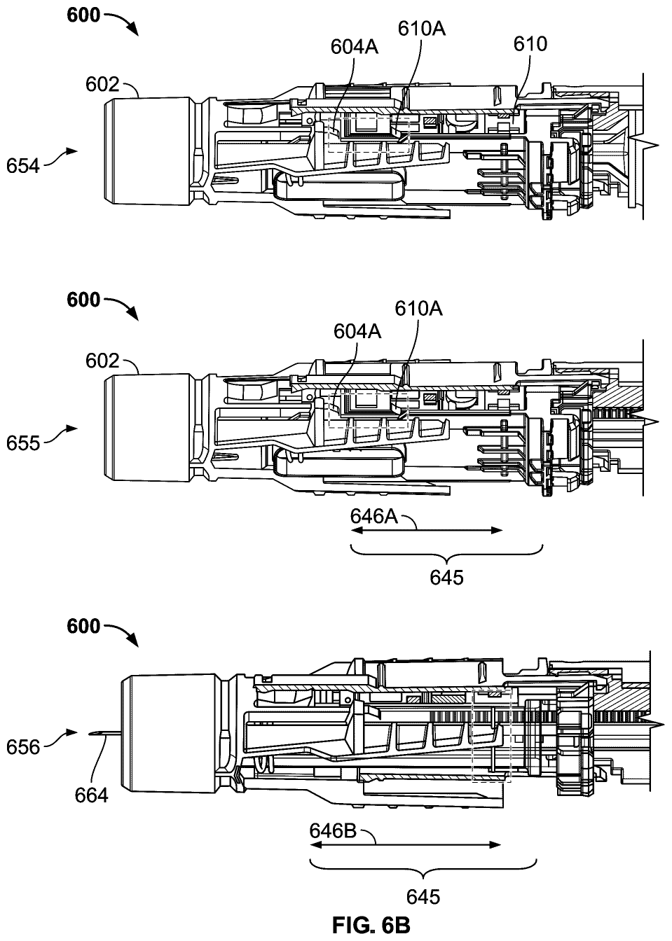

FIGS. 6A-6B illustrate a dispensing movement of an example autoinjector 600. At position 651, a force has been exerted against the needle guard 602 to move the needle guard 602 to a retracted position and expose the needle 664. The needle guard contact 604 includes needle guard contact arm 604A that makes electrical contact with main PCB contact 610A on the main PCB 610 when the needle guard 602 is in the retracted position. The main PCB 610 is housed in the syringe carrier 620. The electrical contact between the needle guard 604 and the main PCB contact 610A generates a signal indicating that the needle guard 602 has been retracted to expose a sufficient length of the needle 664.

At position 652, the needle 664 is exposed through the needle guard 602. The gas canister assembly has been activated to begin releasing pressurized gas into the delivery chamber. The lock ring 630, plunger rod 640, and plunger 644 translate toward the needle 664 to begin dispensing medicament contained in the barrel 662. An aperture 642 in the plunger rod 640 aligns with the optical emitter 672, optical receiver 674, and the optical reflector 676. Light travels from the optical emitter 672 through the aperture 642 to the optical reflector 676. The light is reflected from the optical reflector 676 and then travels through the aperture 642 to the optical receiver 674. The optical receiver 674 generates a signal indicating that the dispensing movement has started. As the dispensing movement continues, the optical receiver 674 can generate additional signals for each subsequent aperture that passes the optical receiver 674 enabling light to transmit from the optical emitter 672 to the optical receiver 674.

At position 653, the plunger rod 640 has reached an end of dose position. The dose progress track 645 includes multiple apertures 642. In the end of dose position, the delivered dose progress 646 is determined based on the number of signals generated by the optical receiver. For example, for a plunger rod that includes six apertures, the optical receiver can generate six signals indicating the passage of each of the six apertures. The end of the dose can be determined by determining that six signals have been received. The number of signals used to determine that a proper end of dose position has been achieved can depend on many factors including, but not limited to, a number of apertures in the plunger rod and a dose volume.

At position 654, the dose has been completed, and the autoinjector 600 has been removed from against the skin of a subject. The needle guard 602 is in an extended position covering the needle 664. The needle guard contact arm 604A is no longer in contact with the main PCB contact 610A indicating that lift off of the autoinjector has occurred. The main PCB 610 can generate a signal indicating the end of the dose based on receiving a specified number of signals from the optical receiver 674 corresponding with translation of the plunger rod 640.

Position 655 illustrates an example failed dose scenario where early or premature lift off has occurred. In this example, the autoinjector 600 has been removed from against the skin of a subject prior to reaching the end of dose. The delivered dose progress 646A indicates that the plunger rod 640 has not been fully depressed. The main PCB 610 has received less than the specified number of optical signals from optical receiver 674. The needle guard 602 is in an extended position. The needle guard contact arm 604A is not in contact with the main PCB contact 610A indicating that the needle 664 is no longer inserted into the skin of the subject. In this example, the main PCB 610 can generate a signal indicating that premature lift off has occurred when the delivered dose progress 646A is less than complete, and the needle guard contact arm 604A and the main PCB contact 610A are no longer in contact with each other.

Position 656 illustrates another example failed dose scenario where a stalled dose has occurred. In this example, the needle guard 602 is in a retracted position. The delivered dose progress 646B indicates that the plunger rod 640 has not been fully depressed, and no additional optical signals are received indicating that the plunger rod 640 has stopped moving. In this example, the main PCB 610 can generate a signal indicating that the dose has stalled. For example, the main PCB 610 can generate a signal indicating that the dose has stalled when less than an expected number of signals is received by the optical receiver and a specified time period has elapsed without receiving additional signals.

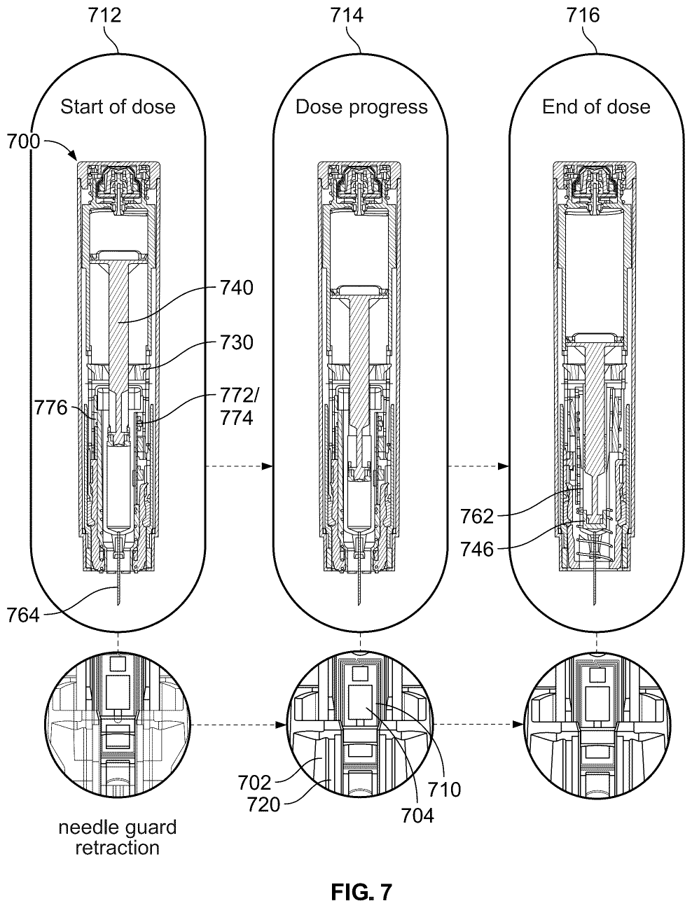

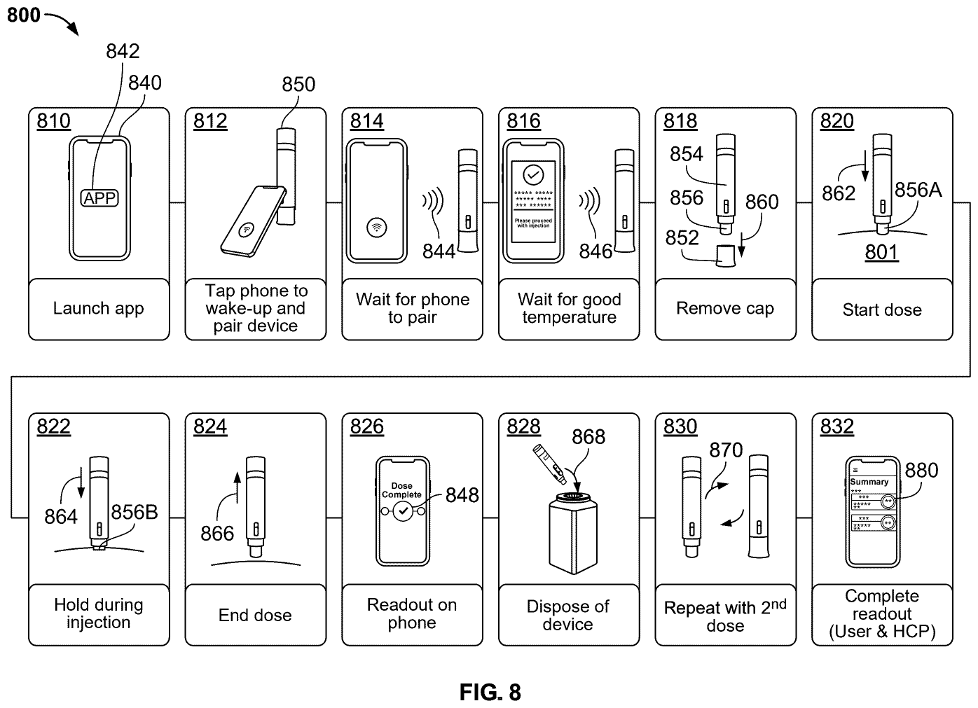

FIG. 7 illustrates a dosing process for an example autoinjector 700. At the start of the dose (712), the needle guard 702 is in a retracted position, and the plunger rod 740 and lock ring 730 shunt toward the needle 764. With the needle guard 702 in the retracted position, the needle guard 702 makes contact with the needle guard switch 704 indicating that the needle guard 702 is retracted. The needle guard switch 704 is on the main PCB 710, which is held in place by the syringe carrier 720. An optical transmission region in the plunger rod 740 passes into the optical path of the optical emitter 772 and the optical receiver 774. The optical transmission region enables an optical signal to be transmitted from the optical emitter 772 through the optical transmission region to the optical reflector 776 and back to the optical receiver 774. The first optical signal received by the optical receiver 774 indicates the start of the dose.

As the dose progresses (714), the plunger rod 740 continues to translate toward the needle 764 in the dispensing movement. As the plunger rod 740 translates, one or more optical transmission regions of the plunger cross the optical path of the optical emitter 772 and the optical receiver 774 generating signals indicating dose progress. During dose progression, the needle guard 702 remains in contact with the needle guard switch 704.

At the end of the dose (716), the plunger 746 reaches the end of the container 762 and the medicament has been fully dispensed. The end of dose can be verified by determining that a specified number of signals were received by the optical receiver 774 during the dispensing movement. The specified number of signals can correspond with the number of optical transmission regions on the plunger rod. For example, if the plunger rod includes five optical transmission regions, then a successful end of dose can be indicated if five optical signals are received by the optical receiver 774 during the dosing process. Any reasonable number of optical transmission regions can be included in the plunger rod (e.g., 1 or more, 2 or more, 3 or more, 4 or more, 5 or more, 10 or more). After the does is completed, the autoinjector 700 can be removed from contact with the subject and the needle guard 702 can extend to cover the needle 764.

FIG. 8 illustrates an example method 800 for using an autoinjector 850. The autoinjector can be similar to, e.g., the autoinjectors 100, 200, 300, 500, 600, and 700 described above. In the method 800, the autoinjector 850 is in communication with a mobile device 840. For example, the autoinjector 850 can be in communication with the mobile device 840 via a wireless transfer protocol module or near field communication protocol (see discussion above). In step 810, the method 800 includes launching an app 842 and/or website on the mobile device 840. In step 812, the method 800 includes tapping the mobile device 840 to wake-up or pair with electronics in the autoinjector 850. The autoinjector 850 may include a physical button to wake the electronics in the autoinjector 850 and pair with the mobile device 840. For example, such a button can be included on the housing of the autoinjector 850. Optionally, activation of a force sensor or electrical contact between elements in the autoinjector 850 can wake up the electronics in the autoinjector 850. The electronics in the autoinjector can include, e.g., a PCB sub-assembly, a voltage sensor, a resistance sensor, an optical transmitter, an optical receiver, a temperature sensor (e.g., a thermistor), a force sensor (e.g., a force sensitive resistor, a load cell, a strain gauge, a force sense capacitor), a vibration sensor (e.g., an accelerometer, a microphone (e.g., a contact microphone or an air microphone), a displacement sensor, a velocity sensor), a switch (e.g., a needle guard switch), and the like. Optionally, the voltage/resistance sensor can sense a movement of a needle guard; the optical receiver can sense a dispensing movement of a plunger rod that includes one or more optical transmission regions, as discussed above; the temperature sensor can sense a temperature in proximity to the medicament; the force sensor can sense an insertion force used to insert the needle of the autoinjector a sufficient distance into the patient; and/or the vibration sensor can sense a dispensing movement of a plunger rod that contacts an audible clicker, as discussed above.

In step 814, the mobile device 840 pairs 844 with the autoinjector 850. Optionally, an indicator light may indicate a status of the autoinjector 850. In step 816, when turned on, the temperature sensor measures a temperature, and the wireless transfer protocol module can communicate 846 the temperature to the mobile device 840. The temperature sensor can provide any number and type of measurements, including a temperature reading or a rate of change of temperature (e.g., 0.01-0.2° C./minute, 0.01-0.1° C./minute, 0.02-0.2° C./minute, 0.02-0.1° C./minute, about 0.05° C./minute, etc.) determined by readings acquired over a time interval (e.g., a time interval of one, two, three, four, or five minutes, in which measurements can be taken every 10, 20, 30, 45, 60, or more seconds). The temperature may be an ambient temperature determined within the autoinjector. While not a direct measurement, the ambient temperature can be used to infer the temperature of the medicament. The temperature may be of a medicament contained within the autoinjector. The user can wait until the medicament is at an appropriate temperature for injection. For example, in many cases, users keep autoinjectors in a refrigerator or other chilled location. After the autoinjector is removed from the refrigerator, the medicament warms up to an appropriate temperature for injection (e.g., 5-10° C., 5-20° C., 5-30° C., 10-20° C., 10-30° C., 20-30° C., less than 30° C., etc.). An indicator on the autoinjector can indicate that the appropriate temperature has been reached. The autoinjector 850 may transmit the temperature of the medicament or the temperature of the ambient environment to the mobile device 840 and the mobile device 340 displays the temperature of the medicament or the temperature of the ambient environment. The autoinjector 850 may transmit a signal to the mobile device 840 indicating that the temperature of the medicament or the temperature of the ambient environment has reached a threshold temperature, and in response, the mobile device 840 displays an indication that the medicament has reached the threshold temperature and/or an indication that the autoinjector is ready for use.

When the medicament is at an appropriate temperature, the autoinjector 850 is ready for use. In step 818, the user can remove 860 a front cap 852 of the ready autoinjector 854. Removing the front cap 852 of the autoinjector 854 reveals the needle guard 856 so that the needle can be inserted into the patient (see discussion below). In step 820, the user brings 862 the needle guard 856 into contact with his or her skin 801 to start the dose. The needle guard 856 is initially in an extended position 856A where the needle is covered. At step 822, the user inserts 864 the needle by pressing the autoinjector further into the skin. Inserting the needle into the patient's skin moves the needle guard 856 into a retracted position 856B. A time delay may be present between a time for inserting the needle into the skin and a time for beginning the dose of medicament. The internal components of the autoinjector inject the medicament into the user when the user inserts the needle into his or her skin (see discussion below). The user holds the autoinjector in place during the injection. In step 824, the dose ends after the medicament has been injected into the user and the autoinjector is removed 866 from the patient. An indicator on the autoinjector can indicate that the dose is complete.

In step 826, after the dose ends, information about the dose can be displayed 848 on the mobile device. For example, information can include whether the dose was successfully injected, the time of the delivery, the amount of medicament delivered, a likely error in delivery (e.g., early lift off, shallow delivery, stalled dose, etc.). The information can be determined based on received signals from, e.g., the force sensor, the temperature sensor, the vibration sensor, the mobile device, the optical receiver.

In step 828, the user can dispose 868 of the autoinjector. The user can dispose of the entire autoinjector. Optionally, the user can remove the electronics from the autoinjector. In some non-limiting examples, the removed electronics can be disposed (e.g., to an appropriate waste stream). In other non-limiting examples, the removed electronics can be returned to the manufacturer for possible reassignment in a controlled manner. In step 830, the user can perform an injection with a second autoinjector by performing 870 the portions of the method 800 described above.

In step 832, the mobile device and/or another device, e.g., a device of a healthcare provider, can provide a full readout of information 880 from the one or more injections performed by the user. For example, the full readout of information can include information about each of the injections performed by the user, including whether each dose was successfully injected, the time of the delivery, the amount of medicament delivered, etc.

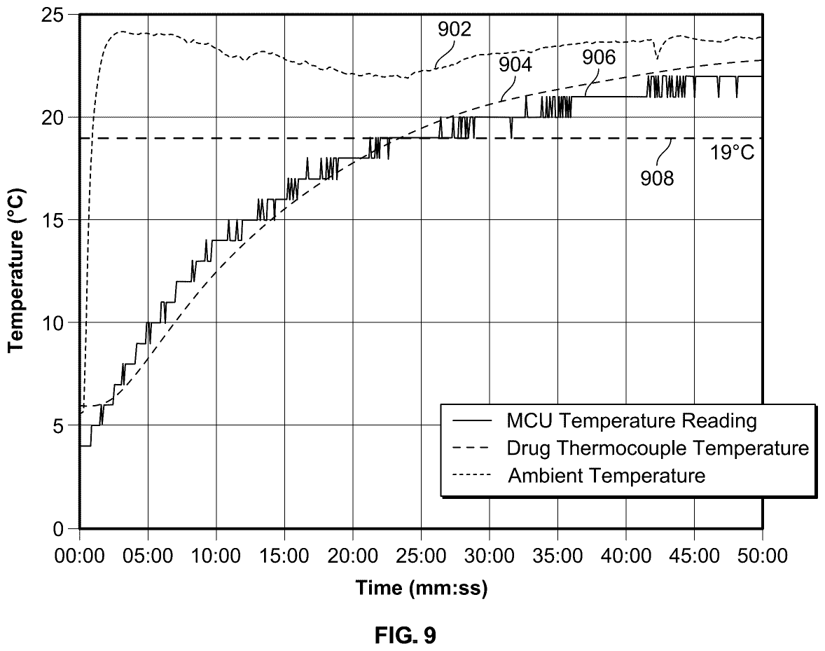

FIG. 9 is plot of temperature versus time. Three temperature traces are shown, ambient temperature 902, temperature of a drug 904 as measured by a thermocouple inserted into the drug, and a temperature reading 906 from a micro-control unit (MCU) with a thermistor. A threshold injection temperature 908 of 19° C. is also shown. When the drug is removed from a cooled location (e.g., a refrigerator), the ambient temperature quickly rises to normal room temperature. The MCU temperature reading 906 tracks the temperature of the drug 904 reasonably well indicating that the MCU temperature reading 906 is an adequate proxy for the temperature of the drug 904.

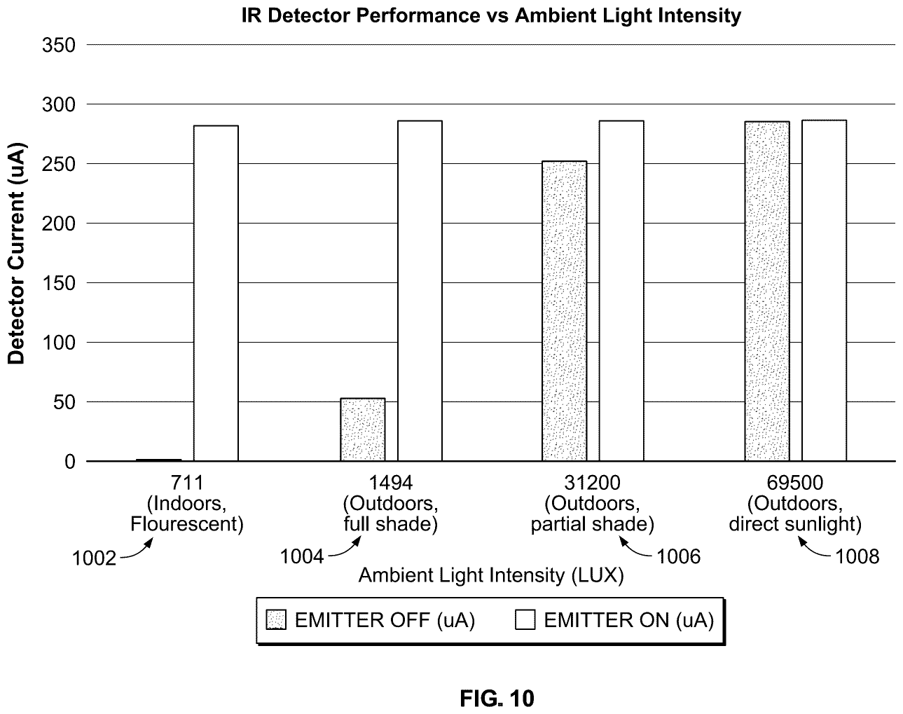

FIG. 10 is a plot of performance of an infrared detector in several lighting scenarios. In an indoor, fluorescent lighting setting 1002, the infrared detector generates minimal current when the emitter is turned off and generates nearly 280 μA (micro amperes) of current. In an outdoor, fully shaded setting 1004, the detector generates nearly 50 μA when the emitter is off and nearly 280 μA when the emitter is on. In an outdoor, partially shaded setting 1006, the detector generates nearly 250 μA when the emitter is off and nearly 280 μA when the emitter is on. In direct sunlight 1008, the detector generates nearly 280 μA when the emitter is in both the on and off positions. This plot shows the importance of optical shielding (e.g., optical shielding described in reference to FIG. 5) to maintain performance of an autoinjector that uses optical signals to detect dose progress. In the most extreme case of direct sunlight 1008, the optical system of the autoinjector would be unable to detect dose progress without shielding because the optical detector is already saturated when the emitter is off.

As used herein, the term “medicament” refers to a pharmaceutical formulation containing at least one active pharmaceutical ingredient (API) which is formulated for administration via injection (e.g., a liquid formulation). The API may be a compound which acts as an anti-viral or has anti-viral properties. The anti-viral API may be a compound that can treat or prevent viral disease such as HIV infection, hepatitis, or other viral disease. The anti-viral API of the medicament may treat or prevent HIV infection alone or in combination with other drugs.

The API may be an anti-HIV drug such as a capsid inhibitor (e.g., an antiretroviral drug that targets the capsid protein shell of viruses). Examples of suitable capsid inhibitors can be found, for example, in U.S. Pat. Nos. 9,951,043; 10,071,985; 11,944,611; 11,787,825; and 12,084,467; and U.S. Publication No.: 20230212148.

The capsid inhibitor may be lenacapavir sodium (SUNLENCA). As used herein, “lenacapavir” (or LEN) refers to N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl) acetamide.

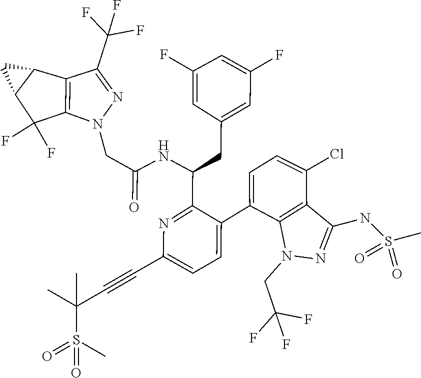 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

### Lenacapavir

Synthesis and characterization of lenacapavir, and salts thereof, are described, for example, in US patent publications US 20180051005 and US 20190300505. Various forms and/or uses of lenacapavir are disclosed, for example, in US 20190083478, US 20190084963, US 20200038389A1, and US 20210188815.

The capsid inhibitor may be lenacapavir, or a pharmaceutically acceptable salt thereof. The capsid inhibitor may be lenacapavir sodium. The capsid inhibitor may be lenacapavir (i.e., the free acid form of lenacapavir).

The present disclosure further includes medicaments (i.e., pharmaceutical compositions) comprising a capsid inhibitor provided herein, or a pharmaceutically acceptable salt thereof, and at least one pharmaceutically acceptable carrier. As used herein, “pharmaceutically acceptable carrier” is meant to refer to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Pharmaceutical compositions of the disclosure are formulated so as to allow the active ingredients contained therein to be bioavailable upon administration of the composition to a patient. Compositions that will be administered to a patient take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of the API, or a pharmaceutically acceptable salt thereof (e.g., for treatment or prevention of an HIV infection or reducing the risk of acquiring HIV).

Examples of suitable excipients are well known to the person skilled in the art of parenteral formulations and can be found, for example, in the Handbook of Pharmaceutical Excipients (eds. Rowe, Sheskey & Quinn), 6th edition 2009.

Examples of excipients in a parenteral formulation (for example, a subcutaneous or intramuscular formulation) include polyethylene glycol. In general, polyethylene glycol (PEG) is a polyether having a general formula H—(O—CH₂—CH₂)ₙ—OH. The PEG may be “capped” by an alkyl group. Optionally, the capped PEG may be of the formula alkyl-(O—CH₂—CH₂)ₙ—O-alkyl (for example, CH₃—(O—CH₂—CH₂)ₙ—OCH₃). The pharmaceutical compositions of the present disclosure can include PEG having an average molecular weight of about 100 to about 1000. The average molecular weight of PEG within the pharmaceutical composition may be about 100 to about 800. The average molecular weight of PEG within the pharmaceutical composition may be about 200 to about 600. The average molecular weight of PEG within the pharmaceutical composition may be about 400. The average molecular weight of PEG within the pharmaceutical composition may be about 300. The average molecular weight of PEG within the pharmaceutical composition may be about 200. In some embodiments of the pharmaceutical composition, different molecular weight PEG can be combined to obtain a desired property or properties (for example, viscosity). Specific examples of PEG include, but are not limited to, PEG 100, PEG 200, PEG 300, PEG 400, PEG 500, and PEG 600. PEG 100, for example, refers to a polyethylene glycol with an average molecular weight of about 100.

The pharmaceutical compositions of the present disclosure can be in the form of a sterile injectable preparation, such as a solution or sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that can be employed are water, Ringer's solution, and isotonic sodium chloride solution. In addition, sterile fixed oils can conventionally be employed as a solvent or suspending medium. For this purpose, any bland fixed oil can be employed, including synthetic mono- or diglycerides. In addition, fatty acids such as oleic acid can likewise be used in the preparation of injectables.

Example formulations suitable for use with the systems and devices of the present application can be found, for example, in U.S. Pat. No. 11,807,625.

The pharmaceutical composition may comprise the API (e.g., lenacapavir or lenacapavir sodium) at a concentration of about 10 mg/mL to about 600 mg/mL. The pharmaceutical composition may comprise the API (e.g., lenacapavir or lenacapavir sodium) at a concentration of about 100 mg/mL to about 400 mg/mL. The pharmaceutical composition may comprise the API (e.g., lenacapavir or lenacapavir sodium) at a concentration of about 250 mg/mL to about 350 mg/mL. The pharmaceutical composition may comprise the API (e.g., lenacapavir or lenacapavir sodium) at a concentration of about 300 mg/mL to about 325 mg/mL. The pharmaceutical composition may comprise the API (e.g., lenacapavir or lenacapavir sodium) at a concentration of about 305 mg/mL to about 310 mg/mL. The pharmaceutical composition may comprise the API (e.g., lenacapavir or lenacapavir sodium) at a concentration of about 309 mg/mL.

The solution provided herein may comprise lenacapavir, or a pharmaceutically acceptable salt thereof, PEG 300, and water. The solution may comprise lenacapavir sodium, PEG 300, and water. The solution may comprise lenacapavir, PEG 300, and water.

The amount of water in the solution comprising lenacapavir sodium, PEG 300, and water may be about 10 w/w % to about 40 w/w %. The amount of water in the solution comprising lenacapavir sodium, PEG 300, and water may be about 15 w/w % to about 35 w/w %. The amount of water in the solution comprising lenacapavir sodium, PEG 300, and water may be about 20 w/w % to about 30 w/w %. The amount of water in the solution comprising lenacapavir sodium, PEG 300, and water may be about 21 w/w % to about 29 w/w %.

The amount of water in the solution comprising lenacapavir sodium, PEG 300, and water may be about 21.1 w/w % to about 27.5 w/w %. The amount of water in the solution comprising lenacapavir sodium, PEG 300, and water may be about 21.13 w/w % to about 27.47 w/w %. The amount of water in the solution comprising lenacapavir sodium, PEG 300, and water may be about 21.1 w/w %. The amount of water in the solution comprising lenacapavir sodium, PEG 300, and water may be about 21.13 w/w %. The amount of water in the solution comprising lenacapavir sodium, PEG 300, and water may be about 23 w/w %. The amount of water in the solution comprising lenacapavir sodium, PEG 300, and water may be about 23.4 w/w %. The amount of water in the solution comprising lenacapavir sodium, PEG 300, and water may be about 23.41 w/w %.

The amount of PEG 300 in the solution comprising lenacapavir sodium, PEG 300, and water may be about 35 w/w % to about 75 w/w %. The amount of PEG 300 in the solution comprising lenacapavir sodium, PEG 300, and water may be about 40 w/w % to about 55 w/w %. The amount of PEG 300 in the solution comprising lenacapavir sodium, PEG 300, and water may be about 43 w/w % to about 47 w/w %. The amount of PEG 300 in the solution comprising lenacapavir sodium, PEG 300, and water may be about 45 w/w %. The amount of PEG 300 in the solution comprising lenacapavir sodium, PEG 300, and water may be about 45.25 w/w %. The amount of PEG 300 in the solution comprising lenacapavir sodium, PEG 300, and water may be about 48 w/w % to about 52 w/w %. The amount of PEG 300 in the solution comprising lenacapavir sodium, PEG 300, and water may be about 50 w/w %. The amount of PEG 300 in the solution comprising lenacapavir sodium, PEG 300, and water may be about 50.1 w/w %. The amount of PEG 300 in the solution comprising lenacapavir sodium, PEG 300, and water may be about 50.13 w/w %.

The amount of lenacapavir sodium in the solution comprising PEG 300 and water may be about 5 w/w % to about 35 w/w %. The amount of lenacapavir sodium in the solution comprising PEG 300 and water may be about 20 w/w % to about 35 w/w %.

The amount of lenacapavir sodium in the solution comprising PEG 300 and water may be about 20 w/w % to about 30 w/w %. The amount of lenacapavir sodium in the solution comprising PEG 300 and water may be about 24 w/w % to about 28 w/w %. The amount of lenacapavir sodium in the solution comprising PEG 300 and water may be about 26.5 w/w %. The amount of lenacapavir sodium in the solution comprising PEG 300 and water may be about 26.46 w/w %.

The amount of lenacapavir sodium in the solution comprising PEG 300 and water may be about 30 w/w % to about 35 w/w %. The amount of lenacapavir sodium in the solution comprising PEG 300 and water may be about 32 w/w % to about 34 w/w %. The amount of lenacapavir sodium in the solution comprising PEG 300 and water may be about 33.6 w/w %. The amount of lenacapavir sodium in the solution comprising PEG 300 and water may be about 33.61 w/w %.

The solution may comprise about 10 w/w % to about 40 w/w % water, about 35 w/w % to about 75 w/w % PEG 300, and about 5 w/w % to about 45 w/w % of lenacapavir sodium. The solution may comprise about 10 w/w % to about 30 w/w % water, about 35 w/w % to about 65 w/w % PEG 300, and about 5 w/w % to about 45 w/w % of lenacapavir sodium.

The solution may comprise about 21.13 w/w % to about 27.47 w/w % water, about 45.25 w/w % to about 58.84 w/w % PEG 300, and about 13.69 w/w % to about 33.61 w/w % of lenacapavir sodium. The solution may comprise about 21.1 w/w % water, about 45.3 w/w % PEG 300, and about 33.6 w/w % of lenacapavir sodium. The solution may comprise about 21.13 w/w % water, about 45.25 w/w % PEG 300, and about 33.61 w/w % of lenacapavir sodium. The solution may comprise about 23.4 w/w % water, about 50.1 w/w % PEG 300, and about 26.5 w/w % of lenacapavir sodium. The solution may comprise about 23.41 w/w % water, about 50.13 w/w % PEG 300, and about 26.46 w/w % of lenacapavir sodium.

The solution provided herein may further comprise ethanol. The solution may comprise about 10 w/w % to about 40 w/w % water, about 20 w/w % to about 75 w/w % PEG 300, about 10 w/w % to about 70 w/w % of lenacapavir sodium, and about 1 w/w % to about 10 w/w % of ethanol. The solution may comprise about 10 w/w % to about 20 w/w % water, about 30 w/w % to about 40 w/w % PEG 300, about 37 w/w % to about 45 w/w % of lenacapavir sodium, and about 3 w/w % to about 8 w/w % of ethanol.

The solution may comprise about 16.93 w/w % water, about 36.22 w/w % PEG 300, about 41.85 w/w % of lenacapavir sodium, and about 5.00 w/w % ethanol. The solution may comprise about 16.9 w/w % water, about 36.2 w/w % PEG 300, about 41.9 w/w % of lenacapavir sodium, and about 5.0 w/w % ethanol.

The medicament which is administered by the injection device of the invention may be a long-acting injectable formulation which can be administered to a patient, for example, twice per month, once per month, once per quarter (e.g., every 3 months), twice per year (e.g., every 6 months), once per year, or less frequently.

Additionally, the medicament can include a formulation which has relatively high viscosity. The medicament may have a viscosity of 100-1000 cP. Preferably, the medicament may have a viscosity of 100-600 cP. For example, the medicament may have a viscosity of 250-500 cP, 150-350 cP or 450-600 cP. The medicament may be a solution comprising lenacapavir sodium the medicament may have a viscosity of 250-500 cP, 150-350 cP or 450-600 cP.

The sensing assembly (e.g., the main PCB of autoinjectors 100, 200, 300, 500, 600, or 700) is intended to include various forms of digital computers, such as printed circuit boards (PCB), processors, digital circuitry, or otherwise parts of a system for determining dosing progression of an autoinjector. Additionally, the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The sensing assembly can include a processor, a memory, a storage device, and an input/output device (for example, sensors). Each of the components is interconnected using a system bus. The sensing assembly is capable of processing instructions for execution within the sensing assembly. The sensing assembly may be designed using any of a number of architectures. For example, the sensing assembly can include a CISC (Complex Instruction Set Computers) processor, a RISC (Reduced Instruction Set Computer) processor, or a MISC (Minimal Instruction Set Computer) processor.

In one implementation, the processor is a single-threaded processor. In another implementation, the processor is a multi-threaded processor. The processor is capable of processing instructions stored in the memory or on the storage device to display graphical information for a user interface on an input/output device.

The memory stores information within the sensing assembly. In one implementation, the memory is a computer-readable medium. In one implementation, the memory is a volatile memory unit. In another implementation, the memory is a non-volatile memory unit.

The storage device is capable of providing mass storage for the sensing assembly. In one implementation, the storage device is a computer-readable medium. In various different implementations, the storage device may be a floppy disk device, a hard disk device, an optical disk device, or a tape device.

The features described can be implemented in digital electronic circuitry, or in computer hardware, firmware, software, or in combinations of them. The features can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM (erasable programmable read-only memory), EEPROM (electrically erasable programmable read-only memory), and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

A number of embodiments have been described. Nevertheless, it will be understood that various modifications may be made without departing from the spirit and scope of the disclosure. Accordingly, other embodiments are within the scope of the following claims.

### Embodiments

The following list provides embodiments of the invention and forms part of the description. These embodiments can be combined in any compatible combination beyond those expressly stated. The embodiments can also be combined with any compatible features described herein:

1. An autoinjector comprising:
a housing;
a needle arranged at a distal end of the housing;
a container disposed within the housing and configured to contain medicament;
a plunger slidably disposed within the container;
a plunger rod configured to push the plunger through the container to dispense the medicament through the needle when the container contains medicament, wherein the plunger rod comprises one or more optical transmission regions;
an optical emitter configured to emit a first optical signal through at least one of the one or more optical transmission regions of the plunger rod; and
an optical receiver configured to receive the first optical signal, wherein a dispensing movement of the plunger rod enables transmission of the first optical signal from the optical emitter through the at least one of the one or more optical transmission regions and to the optical receiver.
   2. The autoinjector of embodiment 1, wherein the first optical signal is indicative of dose progression.
   3. The autoinjector of embodiment 1 or embodiment 2, wherein the container contains the medicament.
   4. The autoinjector of any preceding embodiment, wherein the medicament comprises lenacapavir or a pharmaceutically accepted salt thereof.
   5. The autoinjector of embodiment 4, wherein the medicament comprises lenacapavir sodium.
   6. The autoinjector of embodiment 5, wherein the medicament is a solution comprising: lenacapavir sodium, PEG 300 and water.
   7. The autoinjector of embodiment 6, wherein the solution comprises about 21.13 w/w % water, about 45.25 w/w % PEG 300, and about 33.61 w/w % of lenacapavir sodium.
   8. The autoinjector of embodiment 6, wherein the solution comprises about 23.41 w/w % water, about 50.13 w/w % PEG 300, and about 26.46 w/w % of lenacapavir sodium.
   9. The autoinjector of embodiment 6, wherein the solution further comprises ethanol, optionally wherein the solution comprises about 16.9 w/w % water, about 36.2 w/w % PEG 300, about 41.9 w/w % of lenacapavir sodium, and about 5.0 w/w % ethanol.
   10. The autoinjector of any preceding embodiment, wherein the autoinjector further comprises a gas canister assembly configured to release pressurized gas which, when released, provides a force acting on the plunger rod to push the plunger through the container.
   11. The autoinjector of any of embodiments 1-10, wherein the at least one of the one or more optical transmission regions comprises an aperture disposed within the plunger rod.
   12. The autoinjector of any of embodiments 1-10, wherein the at least one of the one or more optical transmission regions comprises an optically transparent region disposed within the plunger rod.
   13. The autoinjector of any one of embodiments 1-12, wherein the plunger rod is configured to transmit a plurality of optical signals during the dispensing movement of the plunger rod.
   14. The autoinjector of embodiment 13, wherein the plunger rod comprises a plurality of optical transmission regions.
   15. The autoinjector of any one of embodiments 1-14, wherein the first optical signal is configured to indicate completion of the dispensing movement of the plunger rod.
   16. The autoinjector of embodiment 14, wherein a first optical transmission region of the plurality of optical transmission regions is configured to indicate dose start of the medicament, and/or wherein a last optical transmission region of the plurality of optical transmission regions is configured to indicate dose end of the medicament.
   17. The autoinjector of any of embodiments 14 or 16, wherein each optical transmission region of the plurality of optical transmission regions is configured to transmit a corresponding optical signal during the dispensing movement of the plunger rod.
   18. The autoinjector of any one of embodiments 0-17, further comprising:
an audible clicker configured to produce one or more audible clicks during the dispensing movement of the plunger rod.
   19. The autoinjector of embodiment 18, wherein the audible clicker comprises a ring surrounding the plunger rod, and wherein the ring comprises a deflectable protrusion configured to contact the plunger rod and produce the one or more audible clicks.
   20. The autoinjector of embodiment 18, wherein the plunger rod comprises a ridged surface configured to contact the deflectable protrusion of the audible clicker.
   21. The autoinjector of embodiment 20, wherein each ridge of the ridged surface is configured to cause the deflectable protrusion to deflect during the dispensing movement of the plunger rod.
   22. The autoinjector of any one of embodiments 1-21, wherein each of the optical emitter and the optical receiver is, independently, disposed between a proximal end and a distal end of the housing.
   23. The autoinjector of any of embodiments 1-22, wherein the optical emitter comprises an infrared emitter, and/or wherein the optical receiver comprises an infrared receiver.
   24. The autoinjector of any of embodiments 1-23, further comprising an optical reflector configured to provide an optical path between the optical emitter and the optical receiver.
   25. The autoinjector of embodiment 24, wherein the optical reflector is disposed on a first surface within the housing, and wherein the optical emitter and the optical receiver are disposed on a second surface that opposes the first surface.
   26. The autoinjector of any of embodiments 1-25, wherein the optical emitter and/or the optical receiver is disposed on a surface of a printed circuit board.
   27. The autoinjector of any of embodiments 1-26, further comprising a first sensor disposed between a proximal end and a distal end of the housing, wherein the first sensor is configured to detect a first electrical signal generated by applying an insertion force to insert the needle into a user.
   28. The autoinjector of embodiment 27, wherein the first sensor comprises a force sensor.
   29. The autoinjector of any of embodiments 1-28, further comprising a second sensor disposed between a proximal end and a distal end of the housing, wherein the second sensor is configured to detect an ambient temperature in proximity to the container.
   30. The autoinjector of embodiment 29, wherein the second sensor comprises a thermistor.
   31. The autoinjector of any one of embodiments 1-30, wherein the housing comprises an optically shielded housing, and/or wherein the housing further comprises an optically blocking label.
   32. An autoinjector comprising:
a housing;
a needle arranged at a distal end of the housing;
a container disposed within the housing and configured to contain medicament;
a plunger slidably disposed within the container;
a plunger rod configured to push the plunger through the container to dispense the medicament through the needle when the container contains medicament, wherein the plunger rod comprises one or more optical transmission regions;
a mechanism configured so that a dispensing movement of the plunger rod causes the mechanism to generate a first optical signal and transmit the first optical signal through at least one of said one or more optical transmission regions; and
an optical sensor configured to detect said first optical signal generated by the mechanism.
   33. An autoinjector comprising:
a housing;
a needle arranged at a distal end of the housing;
a needle guard configured to expose the needle when an insertion force is applied to insert the needle into a user;
a container disposed within the housing and configured to contain medicament;
a plunger slidably disposed within the container;
a plunger rod configured to push the plunger through the container to dispense the medicament through the needle when the container contains medicament and when the needle is exposed, wherein the plunger rod comprises one or more optical transmission regions;
an optical mechanism configured to emit a first optical signal through at least one of the one or more optical transmission regions of the plunger rod and to receive the first optical signal, wherein a dispensing movement of the plunger rod enables transmission of the first optical signal; and
an electrical mechanism configured to contact a portion of the needle guard, wherein a compression movement of the needle guard generates a first electrical signal.
   34. A system comprising:
an autoinjector of any one of embodiments 1-33; and
a processor configured to process one or more optical signals to track the dispensing movement of the plunger rod.
   35. A method of detecting a dispensing movement of a plunger rod within an autoinjector, the method comprising:
detecting one or more optical signals of an optical emitter as the plunger rod moves during the dispensing movement, wherein the plunger rod comprises one or more optical transmission regions, and
wherein the dispensing movement of the plunger rod provides optical communication between the optical emitter and one or more transmission regions to transmit the one or more optical signals.
   36. A method of detecting a dispensing movement of a plunger rod within an autoinjector, the method comprising:
detecting an optical signal generated due to movement of the plunger rod during the dispensing movement.
   37. The method of embodiment 35 or 36, wherein the plunger rod is configured to transmit a plurality of optical signals during the dispensing movement of the plunger rod.
   38. The method of embodiment 37, wherein the plunger rod comprises a plurality of optical transmission regions.
   39. The method of embodiment 38, wherein a first optical transmission region of the plurality of optical transmission regions is configured to indicate dose start of a medicament, and/or wherein a last optical transmission region of the plurality of optical transmission regions is configured to indicate dose end of the medicament.
   40. The method of any of embodiments 38 or 39, wherein each optical transmission region of the plurality of optical transmission regions is configured to transmit a corresponding optical signal during the dispensing movement of the plunger rod.
   41. The method of any one of embodiments 35-40, wherein the autoinjector further comprises an audible clicker configured to produce one or more audible clicks during the dispensing movement of the plunger rod.
   42. The method of embodiment 41, wherein the audible clicker comprises a ring surrounding the plunger rod, and wherein the ring comprises a deflectable protrusion configured to contact the plunger rod and produce the one or more audible clicks.
   43. The method of embodiment 42, wherein the plunger rod comprises a ridged surface configured to contact the deflectable protrusion of the audible clicker.
   44. The method of embodiment 43, wherein each ridge of the ridged surface is configured to cause the deflectable protrusion to deflect during the dispensing movement of the plunger rod.
   45. The method of any of embodiments 35-44, wherein a first sensor is disposed between a proximal end and a distal end of a housing of the autoinjector.
   46. The method of embodiment 45, wherein the first sensor comprises a voltage sensor or a force sensor.
   47. The method of any of embodiments 45 or 46, wherein the first sensor is disposed on a surface of a printed circuit board.
   48. The method of any of embodiments 45-47, wherein the first sensor is further configured to detect a first electrical signal generated by applying an insertion force to insert a needle into a user.
   49. The method of any of embodiments 35-47, wherein a second sensor is disposed between a proximal end and a distal end of the housing, and wherein the second sensor is configured to detect an ambient temperature in proximity to a container configured to contain a medicament.
   50. The method of embodiment 49, wherein the second sensor comprises a thermistor.
   51. The method of any one of embodiments 35-50, further comprising sending, via a wireless transfer protocol module, signals from a first sensor, if present, and a second sensor, if present, to a mobile device.
   52. The method of embodiment 51, further comprising displaying information about the dispensing movement on the mobile device.
   53. The method of any one of embodiments 35-52, further comprising measuring a temperature of medicament within the autoinjector or an ambient temperature within the autoinjector using a temperature sensor.
   54. The method of embodiment 53, further comprising sending, via a wireless transfer protocol module, signals from the temperature sensor to a mobile device.
   55. The method of any of embodiments 53 or 54, further comprising displaying the temperature of the medicament or the ambient temperature on a mobile device; indicating that the temperature of the medicament or the ambient temperature is above a threshold temperature for use of the autoinjector; or both.
   56. A composition comprising lenacapavir or a pharmaceutically accepted salt thereof for use in the prevention or treatment of HIV, wherein the composition is administered via the autoinjector of any of embodiments 1 to 34.
   57. The composition of embodiment 56, wherein the composition is subcutaneously administered via the autoinjector, optionally wherein the medicament is a solution comprising either:
(a) about 21.13 w/w % water, about 45.25 w/w % PEG 300, and about 33.61 w/w % of lenacapavir sodium; or
(b) about 23.41 w/w % water, about 50.13 w/w % PEG 300, and about 26.46 w/w % of lenacapavir sodium.
   58. The composition of embodiment 56, wherein the composition is intramuscularly administered via the autoinjector, optionally wherein the composition is a solution comprising about 16.9 w/w % water, about 36.2 w/w % PEG 300, about 41.9 w/w % of lenacapavir sodium, and about 5.0 w/w % ethanol.
   59. Use of lenacapavir or a pharmaceutically accepted salt thereof for the manufacture of a medicament for the prevention or treatment of HIV, wherein the prevention or treatment comprises administering the medicament via the autoinjector of any of embodiments 1 to 34.
   60. The use of embodiment 59, wherein the medicament is subcutaneously administered via the autoinjector, optionally wherein the medicament is a solution comprising either:
(a) about 21.13 w/w % water, about 45.25 w/w % PEG 300, and about 33.61 w/w % of lenacapavir sodium; or
(b) about 23.41 w/w % water, about 50.13 w/w % PEG 300, and about 26.46 w/w % of lenacapavir sodium.
   61. The use of embodiment 59, wherein the medicament is intramuscularly administered via the autoinjector, optionally wherein the medicament is a solution comprising about 16.9 w/w % water, about 36.2 w/w % PEG 300, about 41.9 w/w % of lenacapavir sodium, and about 5.0 w/w % ethanol.

1. An autoinjector comprising:
a housing;
a needle arranged at a distal end of the housing;
a container disposed within the housing and configured to contain medicament;
a plunger slidably disposed within the container;
a plunger rod configured to push the plunger through the container to dispense the medicament through the needle when the container contains medicament, wherein the plunger rod comprises one or more optical transmission regions;
an optical emitter configured to emit a first optical signal through at least one of the one or more optical transmission regions of the plunger rod; and
an optical receiver configured to receive the first optical signal, wherein a dispensing movement of the plunger rod enables transmission of the first optical signal from the optical emitter through the at least one of the one or more optical transmission regions and to the optical receiver.

2. The autoinjector of claim 1, wherein the first optical signal is indicative of dose progression.

3. (canceled)

4. The autoinjector of claim 1, wherein the medicament comprises lenacapavir or a pharmaceutically accepted salt thereof.

5. The autoinjector of claim 1, wherein the autoinjector further comprises a gas canister assembly configured to release pressurized gas which, when released, provides a force acting on the plunger rod to push the plunger through the container.

6. The autoinjector of claim 1, wherein the at least one of the one or more optical transmission regions comprises an aperture disposed within the plunger rod.

7. The autoinjector of claim 1, wherein the at least one of the one or more optical transmission regions comprises an optically transparent region disposed within the plunger rod.

8. The autoinjector of claim 1, wherein the plunger rod comprises a plurality of optical transmission regions and the plunger rod is configured to transmit a plurality of optical signals during the dispensing movement of the plunger rod.

9-10. (canceled)

11. The autoinjector of claim 8, wherein a first optical transmission region of the plurality of optical transmission regions is configured to indicate dose start of the medicament, and/or wherein a last optical transmission region of the plurality of optical transmission regions is configured to indicate dose end of the medicament.

12. (canceled)

13. The autoinjector of claim 1, further comprising:
an audible clicker comprising a ring surrounding the plunger rod, and wherein the ring comprises a deflectable protrusion configured to contact the plunger rod to produce one or more audible clicks during the dispensing movement of the plunger rod.

14. (canceled)

15. The autoinjector of claim 13, wherein the plunger rod comprises a ridged surface configured to contact the deflectable protrusion of the audible clicker during the dispensing movement of the plunger rod.

16. (canceled)

17. The autoinjector of claim 1, wherein each of the optical emitter and the optical receiver is, independently, disposed between a proximal end and a distal end of the housing.

18. The autoinjector of claim 17, wherein the optical emitter comprises an infrared emitter, and/or wherein the optical receiver comprises an infrared receiver.

19. The autoinjector of claim 17, further comprising an optical reflector configured to provide an optical path between the optical emitter and the optical receiver.

20. The autoinjector of claim 19, wherein the optical reflector is disposed on a first surface within the housing, and wherein the optical emitter and the optical receiver are disposed on a second surface that opposes the first surface.

21. (canceled)

22. The autoinjector of claim 17, further comprising a first sensor disposed between a proximal end and a distal end of the housing, wherein the first sensor is configured to detect a first electrical signal generated by applying an insertion force to insert the needle into a user.

23. (canceled)

24. The autoinjector of claim 22, further comprising a second sensor disposed between a proximal end and a distal end of the housing, wherein the second sensor is configured to detect an ambient temperature in proximity to the container.

25. (canceled)

26. The autoinjector of claim 1, wherein the housing comprises an optically shielded housing, and/or wherein the housing further comprises an optically blocking label.

27. (canceled)

28. An autoinjector comprising:
a housing;
a needle arranged at a distal end of the housing;
a needle guard configured to expose the needle when an insertion force is applied to insert the needle into a user;
a container disposed within the housing and configured to contain medicament;
a plunger slidably disposed within the container;
a plunger rod configured to push the plunger through the container to dispense the medicament through the needle when the container contains medicament and when the needle is exposed, wherein the plunger rod comprises one or more optical transmission regions;
an optical mechanism configured to emit a first optical signal through at least one of the one or more optical transmission regions of the plunger rod and to receive the first optical signal, wherein a dispensing movement of the plunger rod enables transmission of the first optical signal; and
an electrical mechanism configured to contact a portion of the needle guard, wherein a compression movement of the needle guard generates a first electrical signal.

29. (canceled)

30. A method of detecting a dispensing movement of a plunger rod within an autoinjector, the method comprising:
detecting one or more optical signals of an optical emitter as the plunger rod moves during the dispensing movement, wherein the plunger rod comprises one or more optical transmission regions, and
wherein the dispensing movement of the plunger rod provides optical communication between the optical emitter and one or more transmission regions to transmit the one or more optical signals.

31. (canceled)

32. The method of claim 30, wherein the plunger rod comprises a plurality of optical transmission regions and the plunger rod is configured to transmit a plurality of optical signals during the dispensing movement of the plunger rod.

33. (canceled)

34. The method of claim 32, wherein a first optical transmission region of the plurality of optical transmission regions is configured to indicate dose start of a medicament, and/or wherein a last optical transmission region of the plurality of optical transmission regions is configured to indicate dose end of the medicament.

35-36. (canceled)

37. The method of claim 30, wherein the autoinjector further comprises an audible clicker, the audible clicker comprising a ring surrounding the plunger rod, and wherein the ring comprises a deflectable protrusion configured to contact the plunger rod and produce the one or more audible clicks during the dispensing movement of the plunger rod.

38. The method of claim 37, wherein the plunger rod comprises a ridged surface configured to cause the deflectable protrusion of the audible clicker to deflect during the dispensing movement of the plunger rod.

39-42. (canceled)

43. The method of claim 30, further comprising detecting, by the first sensor, a first electrical signal generated by applying an insertion force to insert a needle into a user.

44-45. (canceled)

46. The method of claim 30, further comprising sending, via a wireless transfer protocol module, signals from a first sensor, if present, and a second sensor, if present, to a mobile device.

47. The method of claim 46, further comprising displaying information about the dispensing movement on the mobile device.

48. The method of claim 30, further comprising measuring a temperature of medicament within the autoinjector or an ambient temperature within the autoinjector using a temperature sensor.

49. The method of claim 48, further comprising sending, via a wireless transfer protocol module, signals from the temperature sensor to a mobile device.

50. The method of claim 48, further comprising displaying the temperature of the medicament or the ambient temperature on a mobile device; indicating that the temperature of the medicament or the ambient temperature is above a threshold temperature for use of the autoinjector; or both.

51. A composition comprising lenacapavir or a pharmaceutically accepted salt thereof for use in the prevention or treatment of HIV, wherein the composition is administered via the autoinjector of claim 1.

52. Use of lenacapavir or a pharmaceutically accepted salt thereof for the manufacture of a medicament for the prevention or treatment of HIV, wherein the prevention or treatment comprises administering the medicament via the autoinjector of claim 1.
